(19) United States
(12) Patent Application Publication
(10) Pub. No.: US 2008/0025875 A1
(43) Pub. Date: Jan. 31, 2008

(54) Chemical, Particle, and Biosensing with Nanotechnology

(76) Inventors: Charles R. Martin, Gainesville, FL (US); Zuzanna S. Siwy, Irvine, CA (US); Punit Kohli, Gainesville, FL (US); Lacramioara Trofin, Murrysville, PA (US); C. Chad Harrell, Redwood City, CA (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/664,339

(22) PCT Filed: Sep. 29, 2005
(86) PCT No.: PCT/US05/35119

§ 371 Date: Sep. 24, 2007

Related U.S. Application Data
(60) Provisional application No. 60/614,784, filed on Sep. 29, 2004.

(51) Int. Cl. C12M 1/34; B01J 19/00; G01N 21/01; G01N 27/00

(52) U.S. Cl. 422/82.01; 422/68.1; 422/82.05; 422/82.09; 435/287.1

(57) ABSTRACT

The subject invention provides novel and efficacious systems and methods for particle, chemical, and/or biocompound sensing. In one embodiment, the system of the invention comprises a sensing device that includes a membrane containing at least one nanochannel that spans all or substantially all of the thickness of the membrane. The nanochannel(s) of the invention can be functionalized to enhance target analyte detection and quantification. In one embodiment, the nanochannel is conically shaped and includes a molecular recognition agent for a target analyte. In certain operations, the sensing systems of the invention quantitatively and qualitatively detect biochemical/biomedical species and biomacromolecules, such as proteins, DNA, cells, spores and viruses, with a high degree of sensitivity and specificity.

### CROSS-REFERENCE TO RELATED APPLICATIONS

This application claims the benefit of U.S. Provisional Application Ser. No. 60/614,784, filed Sep. 29, 2004, the disclosure of which is incorporated herein by reference in its entirety.

### GOVERNMENT SUPPORT

The subject matter of this application has been supported by a research grant from the National Science Foundation, NSF Grant No.: EEC 02-10580 and a research grant from the Defense Advanced Research Projects Agency, DARPA Grant No.: F49620-03-1-0395. Accordingly, the government may have certain rights in this invention.

### FIELD OF THE INVENTION

The invention relates to the field of particle, chemical, and biomolecule sensing. More specifically, the invention relates to sensing devices and methods of detecting and quantifying such compounds with a high degree of specificity and sensitivity.

### BACKGROUND OF THE INVENTION

There are many systems and devices available for detecting a wide variety of analytes in various media. Most of these systems and devices are relatively expensive and require a trained technician to perform the test. Several commercially-available sensing devices include, for example, ion-selective electrodes (e.g., the glass pH electrode); enzyme-based biosensors (e.g., glucose sensors for determining blood glucose concentration), and gene-array and protein-array sensors.

Although currently available sensing devices exhibit some ability to detect certain analytes, they continue to lack the ability to detect analytes with a high degree of sensitivity and/or specificity. In addition to these limitations, there are many analytes for which practical sensing strategies have yet to be developed.

Detection of target analytes using ion-selective electrodes is based on highly selective interactions between a membrane material and a target ion. Unfortunately, such sensors have low detection limits in the micromolar range, as opposed to picomolar range. Furthermore, in many instances, ion-selective electrodes lack the ability to discriminate an ion of interest (e.g., Br⁻) when in the presence of a common interfering ion (e.g., Cl⁻) and is unable to accurately detect and/or quantify the analyte of interest.

Other prior art sensing devices utilize a device that includes a polymer substrate having a metal coating. An antibody-binding protein layer is stamped on the metal-coated substrate. When a target analyte binds to an antibody, a diffraction pattern is generated. A visualization device, such as a spectrometer, is then used to determine the presence of target analytes based on the diffraction pattern. Related sensing devices include an optically active substrate surface that exhibits a first color in the absence of an analyte and a second color in the presence of an analyte. Such sensors have several inherent limitations, the most restraining of which include: requirement of a visualization device or indicator/tag means (see, e.g., gene sensors which require linkage of fluorescent tags to target analytes for detection in gene arrays) to communicate detection of target analyte(s); limited sensitivity; limited specificity; frequent need for recalibration; necessary operator skill; slow and/or false response; and high cost associated with such devices.

Still other sensing technologies are based upon a single nanopore. They are based on monitoring the conductivity of a single nanopore through which a molecule passes through. If the molecule is an ion like DNA, RNA or protein it can be easily “manoeuvred” using an applied electric field across the pore. With an appropriately small pore, the molecule will temporarily create a blockage, thereby reducing or halting the current flow through the pore. The main challenge in developing such nanopore-based sensors is tailoring the sensing materials morphology and the composition at the nanometer scale. Furthermore, it is important that the nanostructured sensing element is manufactured in a form that is amenable to sensing device architectures.

Recently, researchers have shown that biotic (such as protein-based) porous substrates can be manufactured to detect particular chemical and bio-molecular structures. For example, it has been demonstrated that an α-hemolysin protein nanopore can be used as an effective sensing element. Prior publications related to such protein-based porous substrates include, for example, PCT Patent Application Nos. WO96029593 and WO9602937; and J. J. Kasianowicz et al., “Characterization of individual polynucleotide molecules using a membrane channel,” Proc. Natl. Acad. Sci. USA 93, 13770 (1996); M. Akeson et al., “Microsecond Time-Scale Discrimination Among Polycytidylic Acid, Polyadenylic Acid, and Polyuridylic Acid as Homopolymers or as Segments Within Single RNA Molecules,” Biophys. J. 77, 3227 (1999); A. Meller et al., “Rapid nanopore discrimination between single polynucleotide molecules,” Proc. Natl. Acad. Sci. USA 97, 1079 (2000); S. Howorka et al., “Sequence-specific detection of individual DNA strands using engineered nanopores,” Nature Biotechnology, 19, 636 (2001); W. Vercoutere et al., “Rapid discrimination among individual DNA hairpin molecules at single-nucleotide resolution using an ion channel,” Nature Biotechnology 19, 248 (2001); S. Winters-Hilt et al., “Highly Accurate Classification of Watson-Crick Basepairs on Termini of Single DNA Molecules,” Biophys. J. 84, 967 (2003); and S. Howorka et al., “Stochastic Detection of Monovalent and Bivalent Protein-Ligand Interactions,” Angew. Chem. Int. Ed. 43, 842 (2004), all of which are herein incorporated by reference in their entirety.

Unfortunately, biotic nanopore sensing technology is extremely difficult and expensive to produce. For example, with protein-based nanopore sensors, the α-hemolysin nanopore sensing element is imbedded within a very fragile supported lipid bilayer membrane. Even under optimal conditions, such supported lipid bilayer membranes have functional lifetimes of hours, not days or weeks. See, for example, M. Mayer et al., “Microfabricated teflon membranes for low-noise recordings of ion channels in planar lipid bilayers,” Biophysical Journal, 85:2684-2695 (2003), which is herein incorporated by reference in its entirety.

To address the difficulties encountered with biotic nanopore sensors, researchers have recently prepared synthetic nanopore sensors. Such nanopores (also referred to as solid-state nanopores) not only circumvent the problems identified with protein-based nanopores but also address instability problems encountered when working with other forms of biotic nanopores.

There are several methods available for the preparation of solid-state nanopores. Such methods can be divided into two general categories: microfabrication and track etch synthesis methods. Many of these methods, in particular microfabrication methods, require highly specialized and expensive equipment, as well as a high degree of user skill, to prepare such synthetic nanoporous sensors. Moreover, there is no ability to control nanopore size and shape when utilizing such methods. For example, such methods often employ materials that are not water-stable, which can result in formation of nanopores whose size and shape changes over time.

One method for preparing synthetic nanopores employs a feedback-controlled sputtering system and is based on the irradiation of materials with a focused argon ion beam of several keV energy. The preparation of pores in silicon nitride with diameters down to 1.8 nm was demonstrated (see, for example, J. Li et al., “Ion-beam sculpting at nanometre length scales,” Nature 412, 166 (2001), which is herein incorporated by reference in its entirety).

Another method for preparing abiotic nanopore sensing technology combines classical lithographic methods with micromolding of polydimethylsiloxane (PDMS) (see, for example, O. A. Saleh and L. L. Sohn, “An Artificial Nanopore for Molecular Sensing,” Nano Letters 3, 37 (2003), which is herein incorporated by reference in its entirety). This method produces large channels, approximately 200 nm in diameter.

Pores in silicon oxide with diameter of several nanometers can be prepared by electron beam lithography and anisotropic etching in combination with high-energy beam in transmission electron microscope (see, for example, A. J. Storm et al., “Fabrication of solid-state nanopores with single nanometer precision,” Nature Materials 2, 537 (2003), which is herein incorporated by reference in its entirety).

Another method for manufacturing nanochannels in silicon involves reactive plasma etching CHF₃/O₂ (see, for example, J. Han et al., “Entropic Trapping and Escape of Long DNA Molecules at Submicron Size Constriction,” Phys. Rev. Lett. 83, 1688-1937 (1999); D. Stein et al., “Surface-charge-governed ion transport in nanofluidic channels,” Phys. Rev. Lett. 93, 035901 (2004), which are herein incorporated by reference in their entirety). This method provides channels of width down to 70 nm.

Yet another method for preparing nanopores in polymer films utilizes the track-etching technique. With track-etching, polymer foils are irradiated with swift heavy ions of GeV range kinetic energy. The resulting ion tracks are developed by chemical etching. Each individual ion produces a nanometric track typically, but not limited to, ˜10 μm in length (adjustable by beam energy and nature of the material through which the particle passes). Pore densities between a single pore in a membrane and 10¹⁰ pores/cm² can be obtained. Cylindrical or conically shaped pores can be produced e.g., in polycarbonate (PC, Makrofol), polyethylene terephthalate (Hoechst RN 12) (PET), and polyimide (Kapton 50HN, DuPont) (see, for example, P. Apel et al., “Diode-like single ion-track membrane prepared by electro-stopping,” Nucl. Instr. Meth. B 184, 337 (2001); Z. Siwy et al., “Electro-responsive asymmetric nanopores in polyimide with stable ion current signal,” Applied Physics A 76, 781 (2003); Z. Siwy et al., “Rectification and voltage gating of ion currents in a nanofabricated pore,” Europhys. Lett. 60, 349 (2002); Z. Siwy et al., “Preparation of synthetic nanopores with transport properties analogous to biological channels,” Surface Science 532-535, 1061 (2003); C. C. Harrell et al., “Synthetic Single-Nanopore and Nanotube Membranes,” Anal Chem. 75, 6861 (2003); N. Li et al., “Conical Nanopore Membranes. Preparation and Transport Properties,” Anal. Chem. 76, 2025 (2004), which are herein incorporated by reference in their entirety).

One common feature of many current biotic and synthetic nanopores is that they present several pores for sensing, as opposed to a single pore or few pores. Both biotic and synthetic nanopores are often expensive and/or difficult to manufacture as well as fragile by nature. Further, they possess an inability to detect with a high degree of specificity and sensitivity a target analyte. Such nanoporous sensors also lack the ability to qualitatively and quantitatively ascertain analyte presence and concentration. Analyte detection using previously disclosed nanoporous sensors is often determined by the nanoscale of the pores (for example, when a multitude of pores gets blocked by molecules, an observed change in ion current signals detection of the molecules). A nanoporous sensor that utilizes molecular recognition agents to aid in target analyte detection has yet to be developed.

Accordingly, it would be beneficial to provide more accurate, efficient, and simple systems and methods for determining target analyte presence and concentration. It would also be beneficial to provide nano-based sensing systems and methods that can qualitatively and quantitatively detect a wide variety of analytes with a high degree of sensitivity and specificity. Further, systems and methods that utilize an easier sensor platform and/or sensing process; provide sturdy nanoporous materials; enable clear signaling of analyte detection and/or quantification; and are inexpensive and easily manufactured for use in detecting target analyte(s) would be of great benefit.

### SUMMARY OF THE INVENTION

The present invention provides novel sensing systems and methods for particle, chemical, and/or biomolecule/biomacromolecule detection and quantification. The present invention comprises nanosensing structures having a membrane or film that includes a nanochannel that extends at least partially through or entirely through the membrane or film. The membrane or film can be of synthetic substance (e.g., plastic or alumina) or a naturally-occurring material (e.g., protein, biopolymer). In a related embodiment, the present invention further comprises an assay means for translating to the user the detection and/or concentration of the target analyte(s) into an observable signal.

The nanochannel(s) of the invention can be fashioned in any known size or shape to detect a target analyte. The nanochannels can also be either interrupted or uninterrupted. For example, the interior passages of interrupted nanochannel(s) are functionalized with at least one type of molecular recognition agent to facilitate target analyte detection.

In one embodiment, the nanosensing structure of the invention comprises a membrane or film that includes a single, uninterrupted nanochannel that is substantially conical in shape. In other embodiments, the membrane or film contains multiple, uninterrupted nanochannels that are substantially conically-shaped.

In one aspect, the present invention provides a sensing device for detecting a target analyte comprising a membrane or film having at least one nanochannel that extends at least partially through the membrane or film. In one embodiment, target analyte detection is based upon the size and/or shape of the target analyte. Accordingly, the nanochannel(s) in a membrane or film is uninterrupted and constructed and arranged so as to prevent target analyte passage into or through the membrane or film. When a target analyte to be detected blocks the passage of at least one uninterrupted nanochannel, the blockage is transduced (for example, with an assay means) into a signal that is communicated to the user to indicate target analyte detection and/or quantification.

In a related embodiment, a molecular recognition agent is provided, wherein the molecular recognition agent(s) has a high affinity for a target analyte. In one embodiment, a sensing device for detecting a target analyte is provided, wherein the sensing device comprises a membrane or film having at least one functionalized nanochannel with a molecular recognition agent that is specific for the target analyte. Target analyte detection is based upon binding of the target analyte with the molecular recognition agent. When a target analyte to be detected binds to a molecular recognition agent, the binding event and/or nanochannel blockage caused by the binding event is transduced (for example, with an assay means) into a signal that is communicated to the user to indicate target analyte detection and/or quantification.

In one instance, the molecular recognition agent(s) is affixed near an opening to an uninterrupted nanochannel (so that a target and any other analytes could easily pass into and through the membrane or film in which the nanochannel resides), wherein binding of the target analyte to the molecular recognition agent(s) either partially or wholly blocks the opening to the nanochannel. In another instance, the nanochannel(s) is interrupted; the molecular recognition agent(s) is affixed along the inner passage of the nanochannel(s). As with the first instance, upon binding of the molecular recognition agent(s) to a target analyte, the bound analyte partially or wholly blocks the nanochannel(s). Nanochannel blockage is transduced (for example, with an assay means) into a signal that is communicated to the user to indicate target analyte detection and/or quantification.

In another aspect of the invention, a sensing device for detecting a target analyte is provided, wherein the sensing device comprises a membrane or film having at least one functionalized nanochannel with a molecular recognition agent that is specific for an entity bound to the target analyte. Target analyte detection is based upon binding of the entity with the molecular recognition agent. When an entity bound to a target analyte to be detected binds to a molecular recognition agent, the binding event and/or nanochannel blockage caused by the binding event is transduced (for example, with an assay means) into a signal that is communicated to the user to indicate target analyte detection and/or quantification.

In yet another aspect of the invention, a sensing device for detecting a target analyte is provided, wherein the sensing device comprises a membrane or film having at least one functionalized nanochannel with at least one molecular recognition agent that is bound to an entity. Target analyte detection is based upon binding of the target analyte with the molecular recognition agent or the entity, either of which causes disengagement of the entity from the molecular recognition agent. When a target analyte to be detected binds to either a molecular recognition agent or entity, the binding event and/or opening caused by the entity disengagement from the molecular recognition agent is transduced (for example, with an assay means) into a signal that is communicated to the user to indicate target analyte detection and/or quantification.

In one embodiment, the nanochannel(s) in the membrane or film is blocked by the entity. The molecular recognition agent preferentially binds to a target analyte, which is smaller in size than the entity (e.g., binding of molecular recognition agent to target analyte causes the release of the entity and thus opening flow through the nanochannel passage). When a molecular recognition agent preferentially binds to a target analyte, the binding event and/or opening of nanochannel passage caused by the binding event is transduced (for example, with an assay means) into a signal that is communicated to the user to indicate target analyte detection and/or quantification.

In another embodiment, the entity is bound to a first molecular agent affixed to or near the opening of the nanochannel. The entity is also a molecular recognition agent, which preferentially binds to the target analyte. When the entity preferentially binds to a target analyte, the binding event and/or opening of nanochannel passage caused by the binding event is transduced (for example, with an assay means) into a signal that is communicated to the user to indicate target analyte detection and/or quantification.

Another embodiment of the invention provides a sensing device for detecting a target analyte is provided, wherein the sensing device comprises a membrane or film having at least one functionalized nanochannel with at least one molecular recognition agent that has the ability to affect a change in the target analyte. Target analyte detection is based upon the affected change in the target analyte by the molecular recognition agent.

In one embodiment, the molecular recognition agent is an enzyme that is specific for a target analyte. In a related embodiment, the enzyme cleaves or binds target analyte(s) to produce byproducts that are either detectable or are bound to other molecular recognition agents within the nanochannel(s) that are specific for the byproducts. The production of byproducts, binding event(s) (of the byproducts to other molecular recognition agents), and/or nanochannel blockage caused by the binding events are transduced (for example, with an assay means) into a signal that is communicated to the user to indicate target analyte detection and/or quantification.

In yet another aspect of the invention, a sensing device for detecting a target analyte is provided, wherein the sensing device comprises a membrane or film having at least one capped nanochannel, where the nanochannel extends partially through the membrane or film and contains therein a signaling agent. The cap is attached to the nanochannel(s) so as to block the release of the signaling agent. Target analyte detection is based upon the observance of signaling agent(s) released from the nanochannel(s). For example, binding of a target analyte to a molecular recognition agent of the cap or of the nanochannel causes the cap to be detached from the nanochannel, resulting in the release of the signaling agent (that, when detected/observed, communicates to the user that the target analyte is present).

In a method of operation, the subject invention detects a target analyte via the following steps: contacting a nanosensing structure of the invention with a sample; observing a transduction signal, if any (such as an observable signal that is communicated by an assay means); wherein the nanosensing structure comprises a membrane or film having at least one nanochannel that extends at least partially through the membrane or film; and wherein the nanochannel is constructed in accordance with the subject invention as described herein.

According to the present invention, a transduction signal can be one provided by any known strategies demonstrated for other types of sensing systems (such as those described herein). Examples of transduction signals of the invention include, but are not limited to: current (such as ion current) flow; optical signal (such as fluorescence, chemiluminescence, absorbance i.e., infrared, ultraviolet, or visible light using Raman spectrography, or electrogenated chemiluniescence); flow rate or pressure (such as in gas or liquid); or analyte-induced increase or decrease in the concentration of a particular chemical species (such as a hydronium ion-pH sensing).

An advantage of the methods and systems of the present invention is the ability to efficiently detect and quantify various analytes (such as small molecules, macromolecules, biomolecules, and particles) with a high degree of specificity and/or sensitivity (for example, detection limits at sub picomolar regions). For example, analyte species ranging in size from Angstroms to many 10's or even thousands of microns can be detected and quantified using the systems and methods of the invention. The systems and methods of the present invention are particularly advantageous because of the ability to detect and quantify biochemical/biomolecules that are not detectable with currently available nano-based sensors including, but not limited to, the detection of drugs, food additives, anesthetics, peptides, hormones, sugars, proteins, oligonucleotides (such as DNA and RNA), and biological species (such as spores, cells, and viruses).

Further, the nanosensing structures and methods of the present invention may be used in single-element sensors (such as sensors that detect a single type of analyte) or in many-element, array-based sensors (such as sensors that detect a multitude of various analytes).

Other advantages of the present invention include, but are not limited to, an ability to quantitatively as well as qualitatively detect target analyte(s); rapid communication of results (for example, communication of presence (and/or concentration) of target analyte(s) in seconds, or less, under optimal conditions); an ability to analyze a sample without having to label the target analyte(s) in the sample prior to analysis (which can be especially important for biochemical, biological and/or biomedical analytes that are fragile or unstable); relatively low manufacture costs; ease and flexibility of preparation of nanosensing systems, including option for scale-up processing; stable and durable nanosensing structures (including stable nanochannels); ease of operation (for example, low level of operator skill); and little or no need for frequent sensor recalibration.

### BRIEF DESCRIPTION OF THE DRAWINGS

Other objects, features and advantages of the present invention will become apparent upon reading the following detailed description, while referring to the attached drawings, in which:

FIG. 1 shows electron micrographs of large-diameter and small-diameter openings of one embodiment of the present invention.

FIGS. 2A-2F show schematics for a conical nanopore according to several embodiments of the present invention.

FIGS. 3A and 3B show schematics for an electrochemical cell according to another embodiment of the present invention.

FIGS. 3C and 3D show current-time curves for a conical-shaped nanochannel, recorded with two different sample (FIG. 3C where the sample does not contain the target analyte and FIG. 3D where the sample contains the target analyte), according to one aspect of the present invention.

FIG. 4 shows current-voltage (I-V) curves for a gold-lined nanochannel modified with 2-mercaptopropionic acid, recorded at two different pH values, according to one aspect of the present invention.

FIG. 5A shows a current vs. time trace for a conical gold-lined nanochannel with a biotin ligand attached to the gold surfaces prior to exposure to an analyte protein according to another embodiment of the present invention.

FIG. 5B shows a current vs. time trace after exposure to a solution that was 180 pM in an analyte protein according to another embodiment of the present invention.

FIG. 5C shows analogous data in the form of current voltage curves according to another embodiment of the present invention.

FIG. 6 shows current vs. time traces for a conical nanochannel sensor with attached biotin according to another embodiment of the present invention.

FIG. 7 shows a calibration curve of time required for a protein analyte, streptavidin (SA), to shut off the ion-current in a biotin-functionalized nanochannel according to another embodiment of the present invention.

FIG. 8A shows a current vs. time trace for a conical gold-lined nanochannel with a protein G ligand attached to the gold surfaces prior to exposure to an analyte protein horse IgG according to another embodiment of the present invention.

FIG. 8B shows the current vs. time trace according to the embodiment set forth in FIG. 8A.

FIG. 8C shows analogous data of this embodiment in the form of I-V curves.

FIG. 9 shows I-V curves for a conical nanochannel sensor with attached protein G before and after exposure to 10 nM IgG from cat blood according to another embodiment of the present invention.

FIG. 10 shows I-V curves for a biotin-functionalized nanochannel before and after exposure to a solution 10 nM in SA-labeled gold nanoparticles according to another embodiment of the present invention.

FIG. 11 shows current vs. time traces for a sensor according to another embodiment of the present invention after exposure to a solution that was 5 nM in a non-complementary DNA molecule.

FIG. 12 shows analogous data after exposure to a solution that was 5 nM in the DNA that is complementary to the ligand-DNA according to another embodiment of the present invention.

FIG. 13 shows an electron micrograph of an array-pore membrane according to another embodiment of the present invention.

FIG. 14 shows an α-hemolysin protein channel according to one embodiment of the invention.

FIGS. 15A and 15B show methods for immobilizing a molecular recognition agent (such as α-hemolysin protein channel) on a nanosensing system of the invention.

FIG. 16 shows electron micrographs of the surfaces of a membrane containing a nanochannel of the invention.

FIG. 17 shows a schematic of photolithographic process used to draw a molecular recognition agent (such as α-hemolysin protein channel) into a lipid bilayer membrane in one embodiment of the present invention.

### DETAILED DESCRIPTION OF THE INVENTION

The present invention provides sensing systems and methods for detection and quantification of target particles, chemical materials, and/or biological materials (collectively referred to herein as target “analytes”). The present invention comprises nanosensing structures that have a membrane or film that includes at least one nanochannel that extends at least partially through or entirely through the membrane or film. In operation, a sample is placed in contact with a nanosensing structure of the invention and a transduction signal, if any, is observed, wherein the transduction signal indicates target analyte detection and/or quantification.

It is advantageous to define several terms before describing the invention. It should be appreciated that the following definitions are used throughout this application.

As used in the specification and in the claims, the singular form “a,” “an,” and “the” may include plural referents unless the context clearly dictates otherwise. Also, as used in the specification and in the claims, the term “comprising” may include the embodiments “consisting of” and “consisting essentially of.”

As used in the specification and in the claims, the terms “membrane” and “film” are interchangeable. A membrane, as understood by the skilled artisan, is any substrate in which nanochannels of the invention can be fabricated. A membrane of the invention can be of a solid state material or a biological material, or a combination of both solid state and biological materials. In certain embodiments, the membrane of the invention is mechanically, chemically, and electrically stable.

By “biological material” is meant naturally occurring material (e.g., material isolated from a biological environment such as an organism or cell; material otherwise occurring in nature; synthetically manufactured version of a biologically available structure; or a synthetic or non-naturally occurring homologue or derivative of a naturally occurring material that substantially retains the desired biological traits of interest).

The term “solid-state,” as used herein, refers to materials that are not biological materials. Solid-state encompasses both organic and inorganic materials. The material structure can be provided as, e.g., a substrate of inorganic or material, or crystalline material, and is provided as a membrane in which the nanochannel(s) of the invention is to be fabricated. Preferably, a membrane of the invention exhibits any one or combination of the following characteristics: mechanical strength; chemical inertness/stability when subjected to extreme basicity, acidity, and/or aggressive solvents; and flexible/non-brittle.

As used herein, the term “analyte” refers to a particle, chemical or biochemical entity, or biological material to be detected or quantified in accordance with the subject invention. In certain embodiments, an analyte of the invention includes at least one epitope or binding site. With such embodiments, the analyte(s) of the invention can be any substance for which there exists a biotic and/or abiotic molecular recognition agent.

The term “molecular recognition agent,” as used herein, refers to any entity that exhibits high affinity for a target analyte of the invention. In accordance with the subject invention, a molecular recognition agent can specifically bind to a target analyte through chemical, biochemical, and/or physical means. In certain embodiments, the molecular recognition agent(s) is attached to a surface of the nanochannel(s).

The term “uninterrupted nanochannels,” as used herein, refers to nanochannels whose passages are continuous, unbroken, and lack the presence of molecular recognition agents and/or functionalized groups. In contrast, the term “interrupted nanochannels,” as used herein, refers to those nanochannels whose interior passages are non-continuous. For example, interrupted nanochannels of the invention include those nanochannel passages that are intermittently punctuated with molecular recognition agents and/or functionalized groups.

Membranes

Nanochannels of the present invention are synthesized on membranes. Contemplated membranes of the invention include, but are not limited to, carbon, glass, xeolite, silicon, silica, mica, quartz, sapphire, metals (such as gold, silver, nickel, iron, aluminum, and the like), waxes, paraffin films, other polymeric materials (such as polytetrafluoroethylene (PTFE), polyethylene terephthalate, acrylonitrile-butadiene-styrene, acrylonitrile-methyl acetate copolymer, cellophane, ethyl cellulose, cellulose acetate, cellulose acetate butyrate, cellulose propionate, cellulose triacetate, polyethylene, polyethylene-vinyl acetate copolymers, ionomers (ethylene polymers) polyethylene-nylon copolymers, polypropylene, methyl pentene polymers, polyimide (such as KAPTON, DuPont, Circleville, Ohio), polyvinyl fluoride, aromatic polysulfones, as well as any organic polymer and any so-called conductive polymers such as polypyrrole, polyaniline, polythiophene, etc.), biological materials (such as lipid bilayer membranes, cellulosic (plant-based) membranes, protein membranes, or oligonucleotide membranes (such as DNA or RNA)), or a combination thereof.

The membrane of the invention can be selected based on a number of factors including, but not limited to, any one or combination of the following: mechanical strength of the membrane; chemical inertness/stability of the membrane when subjected to extremes of basicity, acidity, and/or aggressive solvents; flexibility/non-brittleness of the membrane; the shelf-life of the membrane; and objective in detecting a target analyte (for example, membrane selection can be based upon the number and/or shape of nanochannel to be synthesized; whether functionalized nanochannels are to be synthesized; the target analyte to be detected, etc.). In certain embodiments of the invention, the membrane is constructed from solid state material. In a related embodiment, the membrane is constructed from a polymeric material. In one embodiment of the invention, the polymeric material is a polycarbonate material.

Synthesis of Nanochannels within Membranes

According to the subject invention, nanochannels are prepared within membranes using any known method for nanopore/nanotube synthesis. For example, nanochannels of the present invention can be synthesized using mechanical, radiological, galvanostatic, electrical, electrochemical, photochemical, or chemical methods. According to the subject invention, the nanochannels of the invention can be non-self-assembled or self-assembled (e.g., self-assembled fullerene-based nanochannels within a membrane). Methods for preparing self-assembled and non-self-assembled nanochannels are discussed in several journals as well as patents including, for example, Kim et al., “Polymeric Self-Assembled Monolayers,” J. Am. Chem. Soc., 117:3963-3967 (1995); Batchelder, “Self-Assembled Monolayers containing Polydiacetylenes,” J. Am. Chem. Soc., 116:1050-1053 (1994); and U.S. Pat. No. 5,885,753, all of which are herein incorporated by reference.

In certain embodiments, nanochannels are synthesized so that they are randomly distributed in the membrane. Other embodiments of the invention provide nanochannels that are arranged in an organized lattice in the membrane.

The membrane is, in one embodiment, selected such that the membrane contains one or more pores that have a selected pore shape. In one embodiment, the membrane includes a single pore having a conical pore shape. As a result, the surface of the substrate includes a larger opening on one side of the membrane and a smaller opening on the opposite side of the membrane. In alternative embodiments, the membrane may include a plurality of pores. It is to be understood that the shape of the pore or pores may be cylindrical, double conical or any other suitable shape.

In certain embodiments, nanochannels of the invention are prepared within membranes via microfabrication methods. One microfabrication method that can be used to prepare nanochannels of the invention is based on the irradiation of materials with a focused argon ion beam of several keV energy and employs a feedback-controlled sputtering system. Another microfabrication method that can be used to synthesize nanochannels of the invention is one that combines classical lithographic methods with micromolding of polydimethylsiloxane (PDMS). Nanochannels of the invention can also be synthesized by spark erosion. Yet another microfabrication method for manufacturing nanochannels within silicon involves reactive plasma etching CHF₃/O₂. Other methods for the microfabrication of nanochannels are described in numerous patents and publications including, for example, U.S. Pat. Nos. 5,300,203; 6,692,717; and 6,756,025.

Additional methods for preparing nanochannels include, but are not limited to, photolithographic or patterning techniques; dip-pen lithography; lithographic stamping; ink-jet printing; or chemical or plasma etching. One method for producing nanochannels within membranes is the “track-etch” synthesis method. With track-etch techniques, a thin film or membrane (also commonly referred to as a “foil”) is tracked or “etched” to form nanochannels. Generally, foils are irradiated with swift heavy ions of MeV to GeV range kinetic energy, which create linear tracks in the film. The resulting ion tracks are developed into nanochannels by chemical etching. Each individual ion produces a nanometric track (or nanochannel) of several tens of micrometers in length (adjustable by beam energy and exposure time). The diameter of the nanochannels are determined by the chemical etch time.

In certain embodiments, nanochannel densities between a single nanochannel in a membrane and 10¹⁰ nanochannels/cm² are synthesized. Nanochannels of the invention can be produced on such membranes as: polystyrene, polystyrene derivatives, silicones, fluoro polymers, polypropylene, polyethylene, poly(meth)acrylic acid, polymethacrylate, polyurethane, polyamide, polycarbonate, polyvinyl chloride, polyvinyl acetate, fluoropolymers, polyethylenes, polypropylene, polyisobutylene, poly(1-butylene), copolymers and blends of the polymers mentioned, alkyl cellulose, hydroxyalkyl cellulose, cellulose ethers, cellulose esters, hydroxypropyl cellulose, hydroxypropyl dextran, hydroxypropylmethyl cellulose, cellulose acetate, carboxyethyl cellulose, cellulose sulphate, dextran sulphate, polyvinyl alcohol, polyethylene oxide, polyvinyl chloride and polyvinylpyrrolidone. In certain embodiments, cylindrical or conically shaped nanochannels of the invention are produced in polycarbonate (PC, Malrofol); polyethylene terephthalate (Hoechst RN 12) (PET); and polyimide (KAPTON, 50HN, DuPont, Circleville, Ohio).

In one embodiment, nanosensing structures of the invention comprise at least one nanochannel synthesized via track-etch method in membranes prepared from polycarbonate and polyester. Such track-etch membranes are available from suppliers such as Osmonics (Minnetonka, Minn.) and Whatman (Maidstone, Kent UK). Such commercially available track-etch membranes contain randomly distributed cylindrical nanochannels of uniform diameter that extend through the entire thickness of the membrane. Nanochannel diameters as small as 10 nm are commercially available at nanochannel densities of up to 10⁹ nanochannels per square centimeter.

In another embodiment, nanosensing structures of the invention comprise at least one nanochannel prepared electronically from aluminum metal. Such porous alumina membranes are commercially available from Whatman (Maidstone, Kent UK). Nanochannel diameters as small as 5 nm can be achieved at nanochannel densities as high as 10¹¹ nanochannels per square centimeter. Membranes can be prepared having the membrane thickness from as small as 5 nm to as large as hundreds of μm.

Other embodiments of the invention comprise at least one nanochannel prepared from membranes such as glass (see R. J. Tonucci et al., “Nanochannel Array Glass,” Science, 258:783-787 (1992)); xeolite (J. S. Beck et al., “A New Family of Mesoporous Molecular Sieves Prepared with Liquid Crystal Templates,” J. Am. Chem. Soc., 114:10834 (1992)), and a variety of other membranes (G. A. Ozin, “Nanochemistry—Synthesis in Diminishing Dimensions,” Adv. Mater., 4:612 (1992)).

In a preferred embodiment, nanochannels are synthesized within a membrane using methods such as those described in German Patent Applications 100 44 565.9 and 102 08 023.2, both of which are incorporated herein by reference in their entirety). FIG. 1 illustrates electron micrographs of large-diameter openings “A” (a large-diameter opening is also referred to herein as the “base” of a nanochannel) and small-diameter openings “B” (a small-diameter opening is also referred to herein as the “tip” of a nanochannel) that are located on opposite “faces” or surfaces of the membrane of an embodiment having a conical nanochannel “C.”

Nanochannels

The present invention enables the preparation of nanochannels of controlled dimensions. Accordingly, a nanochannel can be specifically tailored in shape and/or size based on the sensing objective to be accomplished. Certain embodiments of the subject invention contemplate sensing structures comprising at least one interrupted or uninterrupted nanochannel. In addition, a nanochannel of the invention can be synthesized to extend partially or fully through a membrane. Accordingly, nanochannels of the invention have at least one opening in the membrane.

A nanochannel of the invention can be of any known shape. Additionally, a nanochannel of the invention can be synthesized to be of uniform or variable shape throughout its structure. For example, a nanochannel of the invention can have a cross section (from any axis including vertical or horizontal axis) in the shape of, but not limited to, a polygon (e.g., hourglass, branched polygon, bicentric polygon, concave polygon, convex polygon, cyclic polygon, decagon, equiangular polygon, equilateral polygon, heptagon, hexagon, octagon, pentagon, decogram, octagram, hexagram, nonagram, pentagram, etc.); triangle (e.g., acute triangle, anticomplimentary triangle, equilateral triangle, isosceles triangle, obtuse triangle, right triangle, etc.); parallelogram (e.g., equilateral parallelogram, rectangle, rhomboid, etc.); Penrose tile (e.g., Penrose dart, Penrose kite, etc.); circle (e.g., Archimedes' circle, Bankoff circle, circumcircle, excircle, incircle, nine point circle, etc.); lune; semicircle; and ellipse.

A nanochannel of the invention can be of any size including, for example, from nanometric dimensions to micrometric dimensions. A nanochannel of the invention can also be of uniform or variable size throughout the nanochannel. Further, the depth to which a nanochannel of the invention extends in and/or through a membrane depends on the intended sensing application. The depth (or length) of the nanochannel can be controlled by varying the thickness of the membrane and/or during nanochannel synthesis. Accordingly, the depth of at least one nanochannel in a membrane can be in the range from less than 1 nm to many hundreds of nm's.

Where a nanochannel of the invention has more than one opening, the openings can be identical or dissimilar in size and/or shape. In one embodiment, a nanochannel is of conical shape, with a tip opening and a base opening, where the dimensions of the tip and base openings are not identical. In other embodiments, the tip and base openings of a conically-shaped nanochannel have identical dimensions (e.g., where the nanochannel is of cylindrical or hourglass shape).

According to the subject invention, the diameter of at least one opening of a nanochannel can be in the range from less than 1 nm to larger than many hundreds of nm's, depending on the size of the analyte species to be detected. For some embodiments, it will be preferred that the diameter of at least one nanochannel opening does not exceed 1.0 nm. For other embodiments, it will be preferred that the nanochannel opening diameter does not exceed 10 nm. Yet with other embodiments, it will be preferred that the nanochannel opening diameter does not exceed 100 nm. For further embodiments, it will be preferred that the nanochannel opening diameter does not exceed 10 μm. With other embodiments of the invention, it is preferred that the nanochannel opening diameter be at least 100 nm, 200 nm, 300 nm, 400 nm, 500 nm, 600 nm, 700 nm, 800 nm, or 900 nm.

In one embodiment, where the analyte to be detected is a small molecule having a diameter of about 0.5 nm, the tip opening might be manufactured to have a diameter of about 1 nm. In another example, where the target analyte to be detected is a biological material (such as a spore or pollen) with diameter of 1 μm, the tip opening might be manufactured to have a diameter of about 2 μm.

Functionalization of Nanochannels

In certain embodiments, detection of a target analyte is solely a function of the shape and/or size of the nanochannel(s). In other embodiments, a nanochannel has different functionalized groups to aid in target analyte detection. Methods used to functionalize a nanochannel surface depend on the composition of the membrane and are well known in the art.

For example, functionalization of nanochannel(s) in silica membrane is accomplished using silane chemistry. A reactive functional group can be attached to surfaces of a nanochannel by reacting a hydrolytically unstable silane that contains the functional group with surface silanol sites on the nanochannel to obtain covalent, oxygen/silicon bonds between the surface and the silane. The reactive functional group can be used to attach a biochemical molecule (such as a molecular recognition agent) to the surface bound silane. In this way the nanochannel surface is functionalized with the molecular recognition agent.

The surface of nanochannel(s) in a polymeric membrane can be functionalized with a metal to coat the nanochannel walls with this metal using well-known chemical, or other, methods. The metal surface can then be functionalized by chemical entities that contain the thiol function group. Thiolate chemistry enables using various derivatives without significant alteration of preparation procedure of nanochannels. There is a substantial variety of commercially available thiols with —OH, —SH, —COOH, and NH₂ functional groups. Thiols that can be used in accordance with the subject invention to prepare functionalized nanochannels include, but are not limited to, thioglycerol (—OH); mercaptosuccinic acid (—COOH), thioglycolic acid (—COOH), and 1-amino-2-methyl-2-propanethiol (—NH₂) (the terminal function group is indicated in parenthesis). The selection of a specific terminal functional group is generally governed by the conjugation reactions planned for the nanochannel(s). Should it be necessary to vary the thiol structure, for instance the hydrocarbon chain length, one can synthesize the compound series by using classical methods of organic chemistry.

In certain embodiments, functional groups can be introduced by copolymerization. Natural amino acids are chemically similar to lactic acid but offer a variety of functional groups on their side-chains (—OH, —COOH, —NH₂, —SH, etc.). Moreover, like lactic acid, amino acids are found in all cell types, so that the polymer degradation products are non-toxic. Monomers derived from an amino acid and lactic acid can be synthesized by standard methods and used for random copolymerization with lactide.

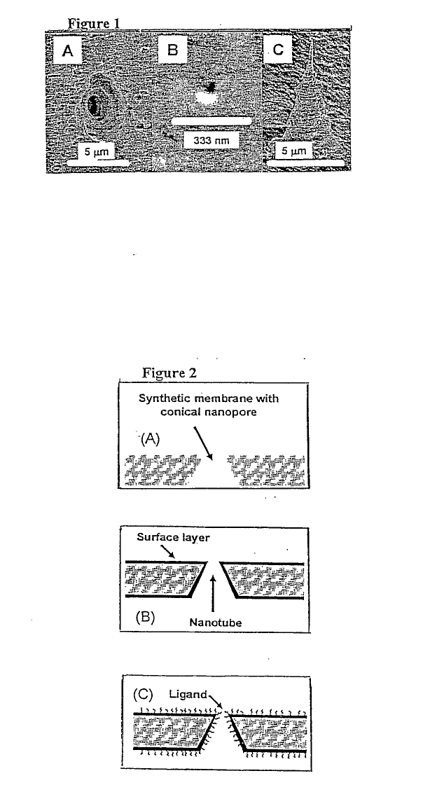

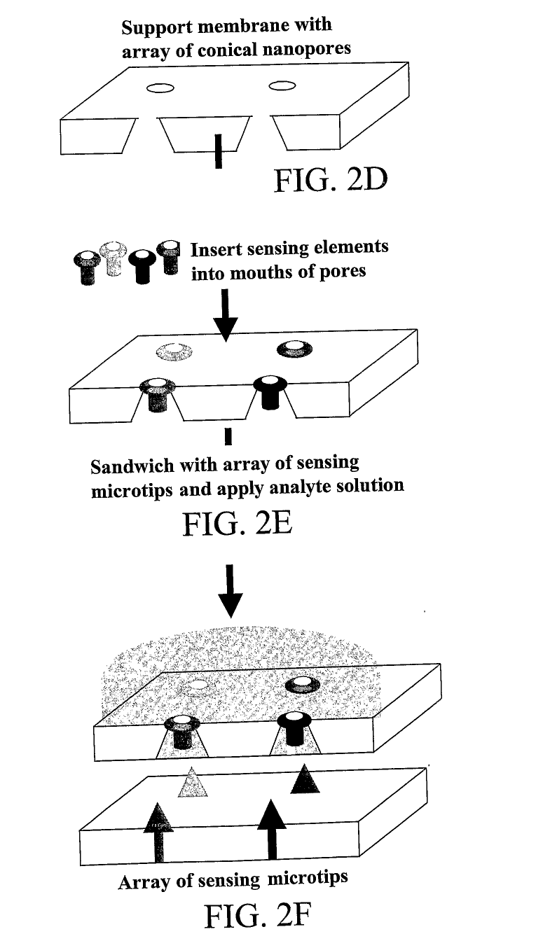

In some embodiments, it may be beneficial to functionalize nanochannel(s) by lining nanochannel surfaces with a lining material (see FIG. 2). One advantage of lining the nanochannel(s) of the invention is the ability to manipulate the dimension and/or shape of the nanochannel opening(s) in accordance with the analyte to be detected. Another advantage to lining the nanochannel(s) of the invention is that the lining material may be more receptive to methods for attaching molecular recognition agents to the nanochannel(s) than the membrane.

Examples of lining materials that may be used in the present invention include, but are not limited to, polymers (such as polytetrafluoroethylene (PTFE), polyethylene terephthalate, acrylonitrile-butadiene-styrene, acrylonitrile-methyl acetate copolymer, cellophane, ethyl cellulose, cellulose acetate, cellulose acetate butyrate, cellulose propionate, cellulose triacetate, polyethylene, polyethylene-vinyl acetate copolymers, ionomers (ethylene polymers) polyethylene-nylon copolymers, polypropylene, methyl pentene polymers, polyimide (such as KAPTON, DuPont, Circleville, Ohio), polyvinyl fluoride, aromatic polysulfones, as well as any organic polymer and any so-called conductive polymers such as polypyrrole, polyaniline, polythiophene, etc.); semiconductor materials (such as, but not limited to, TiS₂, TiO₂, silicon, germanium, etc.); carbon-based materials (such as, but not limited to, graphite or various forms of graphitic carbon); metals (such as, but not limited to, gold, silver, copper, iron, steel, chromium, nickel, platinum, aluminum, copper, chromium oxide, zirconium etc.); other inorganic materials (such as, but not limited to, SiO₂, Li⁺, etc.); or a combination thereof.

In one embodiment, lining of a nanochannel is accomplished by lining the nanochannel with a polymer. Polymer lining of the nanochannel(s) of the invention can be accomplished using many substances that are composed of monomer units. “Monomer units” are the individual moieties that are repeated to form “polymers.” Multiple monomer units are covalently attached when in the form of a backbone of a polymer. Polymers that are made from at least two different types of monomer units are referred to as “copolymers.” Polymerizing or copolymerizing describes the process by which multiple monomers are reacted to form covalently linked monomer units that form polymers or copolymers, respectively. A discussion of polymers, monomer units, and the monomers which they are made may be found in Stevens, Polymer Chemistry: An Introduction, 3^{rd} ed., Oxford University Press, 1999, the contents of which are incorporated by reference.

Methods employed for polylactide synthesis allow for polymer surface functionalization, which can be used when lining nanochannels of the invention with a polymer. Polymerization occurs by an insertion mechanism mediated by Lewis acids such as Sn²⁺ whose bonds with oxygen have significant covalent character. An alcohol complexed with the metal ion initiates polymerization, which continues by stepwise ring-opening of the lactide monomers to generate a new alkoxide-metal complex capable of chain growth. The polymer molecular weight can be controlled by the molar ratio of initiating alcohol to the lactide monomer. The resulting polyester possesses directionality with a hydroxyl terminus (from the final monomer) and a functional group at the ester terminus determined by the structure of the initiating alcohol. The latter can contain a variety of functional groups.

Other chitosan, PEGylated PLGA (poly(lactic-co-glycolic acid)) and other PEGylated compounds. For example, a commercially available PEG-maleimide can be incorporated into the chain-end thiols on the outer surfaces of the nanochannels. “PEGylated” compounds are compounds modified by attaching PEG (polyethylene glycol) chains to the compounds.

In another embodiment, the surface of nanochannel(s) in an aluminum membrane can be functionalized by lining the surface of the nanochannel(s) with a first layer of polyethylene imine (PEI) and a second layer of a biotinylated PEG.

Nanocap-Nanochannel Assembly

In certain embodiments, nanochannels of the invention comprise a void in which a signaling agent resides. Such nanochannels are preferably functionalized to include nanoparticles (also referred to herein as “nanocaps”) that act as a cap over at least one opening of the nanochannel to block the release of the signaling agent present within the void. According to the subject invention, the nanocap provides a mechanism by which the signaling agent can be selectively released when in the presence of a target analyte. Release of signaling agent(s) upon target analyte detection is achieved by linking a molecular recognition agent to the uncapping/discharge mechanism of the nanocap-nanochannel assembly.

In one embodiment of the invention, a sensing system is provided in which a single nanochannel or multiple nanochannels are provided on a membrane substrate. Blocking an opening of the nanochannel(s) is at least one nanoparticle, which prevents current ion flow through the nanochannel(s). In a related embodiment, the nanoparticle(s) are attached to the tip opening of the nanochannel(s) using a molecular recognition agent (for example, a selective receptor or ligand such as biotin). The molecular recognition agent is attached to a surface of the nanochannel using known methods; attachment occurs preferably on the surface near the tip opening of the nanochannel. A nanoparticle is then prepared that has the target analyte to be detected attached (such as Streptavidin) to the nanoparticle surface. In a preferred embodiment, the diameter of the nanoparticle is slightly larger than the opening tip diameter of the nanochannel. In certain embodiments, the nanocap is a polymer or biopolymer, or if the opening of the nanochannel is small enough, even a molecule.

To block the tip opening of the nanochannel with a nanoparticle, the nanochannel surface that contains the molecular binding agent is exposed to the analyte-tagged nanoparticle. The analyte on the nanoparticle binds to the molecular recognition agent, effectively blocking the opening of the nanochannel. When the membrane is then exposed to a solution containing the target analyte, the analyte in the solution competes with the analyte attached to the nanoparticle. As a result of this competition, the analyte from the solution displaces the nanoparticle by binding to the molecular recognition agent(s) that held the nanoparticle in place. Since the analyte displaces the nanoparticle, the opening of the nanochannel is effectively open to allow for a transmembrane ion current (detection of ion-current flow indirectly indicates presence and/or concentration of the target analyte).

The method for attaching nanocaps, as described herein, can be applied to a variety of sensing systems of the invention. For example, nanocap attachment using molecular binding agents and analyte-tagged nanocaps can be applied to nanochannels of any shape or variety. In one embodiment of the invention, the method for attaching a nanocap as described above is applied to nanochannels that are conical or cylindrical in shape. Further, the nanoparticle can be attached to any single or multiple openings of a nanochannel. For example, the nanoparticle can be attached not only to a tip opening but also a base opening of the nanochannel.

In another embodiment of the invention, the molecular recognition agent is provided on a surface within the nanochannel as opposed to near an opening of the nanochannel. When the nanochannel is exposed to a target analyte, the analyte binds to the molecular recognition agent. A nanoparticle is provided wherein the same or different molecular recognition agent is affixed to the nanoparticle. The nanochannel with the bound analyte-molecular recognition agent is then exposed to the nanoparticle that contains a molecular recognition agent to the analyte. Since the analyte has been bound to the nanochannel surface, when nanoparticle-molecular recognition agent complex binds to the analyte, the nanoparticle is effectively bound to the nanochannel as well. Preferably, the nanoparticle blocks an opening of the nanochannel, which in turn prevents ion current flow through the nanochannel that is detectable. In a related embodiment, the nanoparticle is detectable (such as fluorescent compound), where an increase in detectable nanoparticles (such as an increase in fluorescent intensity) at the nanochannel membrane substrate indicates analyte detection and/or concentration.

In yet another embodiment of the invention, a surrogate compound, as opposed to the target analyte, is attached to the nanocap. Preferably, the surrogate compound is a compound that does not bind as strongly to the molecular recognition agent affixed to the surface of the nanochannel(s). Examples of surrogate compounds include, but are not limited to, a metabolite or chemically related compound of the target analyte.

In a related embodiment, in addition to tagging the nanocap with an analyte or surrogate compound, a detectable compound (such as a fluorescent agent) is attached to the nanocap(s). Alternatively, the nanoparticle itself is a detectable compound.

In certain embodiments, after sensing a target analyte with a molecular recognition agent, a nanocap of the invention can be uncapped or discharged from a nanochannel through the use of energy-bearing biomolecular motors such as, but not limited to, the actin-based system (Dickinson, R. B. and Purich D. L., “Clamped-filament elongation model for actin-based motors,” Biophys. J., 82:605-617 (2002), which is herein incorporated by reference in its entirety).

A nanoparticle can be attached over an opening of a nanochannel by covalent bonds. For example, silica nanoparticles can be linked by disulphide bonds to nanochannels formed in silica membrane. Initially, the surface at the ends of silica nanochannels is functionalized with an —SH linker. If necessary, the inner surfaces of the nanochannels are protected with, for example, a silane group such as (Me—O)₃—(CH₂)₃—OH. After the protection step, the silica surface layers at the nanochannel openings are removed to expose fresh silica. The freshly-exposed silica will be reacted with the silane, such as (Me—O)₃—Si—(CH₂)₃—SH to attach the requisite —SH linker to the openings of the nanochannels. The length of the alkyl chain in a silane can be varied to allow placement of the —SH linker at any desired distance from the nanochannel opening. These —SH functionalities are then reacted with pyridine disulfide in order to obtain nanochannels with an activated disulfide bond at the openings of the nanochannels. Hence, nanochannels with an activated disulfide at their openings and nanocaps with an —SH group on their surface are available for linkage through disulfide bond formation.

Similar functionalization methods are applicable to nanochannels and nanoparticles prepared from materials other that silica, including biodegradable polymers. For example, the biodegradable polymer polylactide can be prepared using a sulfur-containing initiator to produce an initiator-derived protected thiol group at the ester terminus of the chain. This group can be used to attach a nanoparticle cap via disulfide chemistry. Alternatively, higher —SH density can be achieved using the brush polymer approach to incorporate additional thiol groups (see, for example, Hrkach, J. S. et al., “Synthesis of Poly(L-Lactic acid-co-L-lysine) Graft Copolymers,” Macromolecules, 28:4736-4739 (1995), which is herein incorporated by reference in its entirety).

Other types of covalent bonds, for example amide and ester bonds, can be used to attach a nanoparticle over an opening of a nanochannel. For example, siloxane-based linking can be used. This would be particularly useful when the cap is composed of silica as the silanol sites on the silica surface react spontaneously with siloxanes to form a covalent oxygen-silicon bond.

For metal nanochannels (or metal lined nanochannels) or nanocaps, thiol linkers can be used for attachment. For example the molecule (Me—O)₃—Si—(CH₂)₃—SH could be attached to a silica membrane surrounding a nanochannel opening and a gold nanocap attached by using the —SH end of this molecule ((Me—O)₃—Si—(CH₂)₃—SH). It is well known that such thiols form spontaneous As—S bonds with gold surfaces.

In another method of capping, nanoparticles can be electrophoretically placed within the mouths of nanochannels so that the entire mouth of the nanochannel is blocked when disulfide bonds are formed between the wall of the nanochannel and the nanoparticle as described in Miller, S. A. and Martin, C. R. “Electroosmotic Flow in Carbon Nanotube Membranes,” J. Am. Chem. Soc., 123(49):12335-12342 (2001), which is herein incorporated by reference in its entirety.

By way of example, a nanochannel-containing membrane is mounted in a U-tube cell with platinum electrodes immersed into the buffer solution on either side of the membrane. The —SH-functionalized nanoparticles are added to the cathode half-cell. The buffer solution is maintained at pH=7 so that a small fraction of the —SH groups on the nanoparticles are deprotonated. These negatively charged particles are driven into the openings of the nanochannels electrophoretically by using the Platinum electrodes to pass a constant current through the membrane. Hence, the electrophoretic force causes the nanoparticles to nestle into the nanochannel openings, where disulfide bond formation will occur. Once nestled this way, chemical bonds can be formed between the cap and the nanochannel. As discussed above, these chemical bonds can be disrupted by the analyte to release the cap from the nanochannel.

As an alternative to the electrophoretic assembly method, —SH labeled nanocaps can be suspended in solution together with the activated disulfide labeled nanochannels. Here, the nanocaps can spontaneously self-assemble to the nanochannels.

In addition to —SH linking, other covalent linking methods can be used to link nanochannels and nanoparticles. Non-covalent linking methods can also be used. These include, for example, DNA hybridization (see, for example, Mirkin, C. A. “Programming the Self-Assembly of Two and Three-Dimensional Architectures with DNA and Nanoscale Inorganic Building Blocks,” Inorg. Chem., 39:2258-2272 (2000), which is incorporated herein by reference in its entirety); the biotin/avidin interaction (see, for example, Connolly, S. & Fitzmaurice, D., “Programmed Assembly of Gold Nanocrystals in Aqueous Solution,” Adv. Mater., 11:1202-1205 (1999), which is herein incorporated by reference in its entirety); and antigen/antibody interactions (Shenton, W. et al., “Directed Self-Assembly of Nanoparticles into Macroscopic Materials Using Antibody-Antigen Recognition,” Adv. Mater., 11:449 (1999), which is herein incorporated by reference in its entirety). With certain nanocaps linked over nanochannel openings by covalent bonds, the uncapping/discharge mechanism can include bond cleavage by a specific enzyme, for example, a hydrolase enzyme.

Alternatively, the nanocap can be linked to a nanochannel by hydrogen bonding or by acid and/or basic sites on the nanochannel. With such nanocap-nanochannel assemblies, uncapping can be achieved by a change in the pH of the surrounding medium.

Other nanocaps of the invention can contain an energy sensitive inert gas that could be used to trigger release of the signaling agent from the nanochannel.

According to the subject invention, nanocaps of the invention have an outside diameter slightly larger than the inside diameter of nanochannels of the invention. Depending on the application, the nanocap of the invention can be prepared from the same material as the membrane in which the nanochannel resides or, alternatively, is prepared from a different material or combination of different and same material as the membrane. Methods of preparation of nanoparticles are well known in the art. For example, the preparation of monodisperse sol-gel silica nanospheres using the well-known Stober process is described in Vacassy, R. et al., “Synthesis of Microporous Silica Spheres,” J. Colloids and Interface Science, 227:302 (2000), which is incorporated herein by reference in its entirety.

Signaling Agents

The nanocap-nanochannel assemblies of the invention can use many different signaling agents or combination of different signaling agents. In certain embodiments, a nanocap-nanochannel assembly uses at least one signaling agent with at least one nanochannel. The present invention includes the use of nanocap-nanochannel assemblies that can release more than one signaling agent that are separately contained within multiple nanochannels to indicate detection of and/or concentration of different types of target analytes.

Signaling agents of the invention include, but are not limited to, chromagens, chemiluminescers, radioactive labels, dyes that can be detected by optical absorbance, fluorophor molecules, quantum dots, redox-active molecules, as well as species that cause the pH of the solution to change raman-active substances. Chromagens include compounds which absorb light in a distinctive range so that a color may be observed, or emit light when irradiated with light of a particular wavelength or wavelength range (e.g., fluorescers). Examples of chromogens as signaling agents include, but are not limited to, colloidal particles (i.e., nanometer scale gold or magnetic-polymer composites); dyes (i.e., quinoline dyes, triarylmethane dyes, acridine dyes, alizarin dyes, phthaleins, insect dyes, azo dyes, anthraquinoid dyes, cyanine dyes, phenazathionium dyes, and phenazoxonium dyes, UV absorbers, IR absorbers, raman-active substances); and fluorescent compounds including, but not limited to fluorescent compounds having primary functionalities (i.e., 1- and 2-aminoaphthalene, p,p′-diaminostilbenes, pyrenes, quaternary phenanthridine salts, 9-aminoacridines, p,p′-diaminobenzophenone imines, anthracenes, oxacarbocyanine, merocyanine, 3-aminoequilenin, perylene, bis-benzoxazole, bis-p-oxazolyl benzene, 1,2-benzophenazin, retinol, bis-3-aminopyridinium salts, hellebrigenin, tetracycline, sterophenol, benzimidazolylphenylamine, 2-oxo-3-chromen, indole, xanthene, 7-hydroxycoumarin, phenoxazine, salicylate, strophanthidin, porphyrins, triarylmethanes and flavin) and fluorescent compounds having functionalities for linking or can be modified to incorporate such functionalities (i.e., dansyl chloride, fluoresceins such as 3,6-dihydroxy-9-phenylxanthhydrol, rhodamineisothiocyanate, N-phenyl 1-amino-8-sulfonatonaphthalene, N-phenyl 2-amino-6-sulfonatonaphthalene, 4-acetamido-4-isothiocyanatostilbene-2,2′-disulfonic acid, pyrene-3-sulfonic acid, 2-toluidinonaphthalene-6-sulfonate, N-phenyl, N-methyl 2-aminonaphthalene-6-sulfonate, ethidium bromide, atebrine, auromine-0, 2-(9′-anthroyl)palmitate, dansyl phosphatidylethanolamine, N,N′-dioctadecyl oxacarbocyanine, N,N′-dihexyl oxacarbocyanine, merocyanine, 4-(3′-pyrenyl)butyrate, d-3-aminodesoxyequilenin, 12-(9′-anthroyl)stearate, 2-methylanthracene, 9-vinylanthracene, 2,2′-(vinylene-p-phenylene)-bis-benzoxazole, p-bis[2-(4-methyl-5-phenyloxazolyl)]benzene, 6-dimethylamino-1,2-benzophenazin, retinol, bis(3′-aminopyridinium) 1,10-decandiyl diiodide, sulfonaphthylhydrazone of hellebrigenin, chlortetra-cycline, N-(7-dimethylamino-4-methyl-2-oxo-3-chromenyl)maleimide, N-[p-(2-benzimidazolyl)-phenyl]maleimide, N-(4-fluoranthyl) maleimide, bis(homovanillic acid), resazarin, 4-chloro-7-nitro-2.1.3-benzooxadiazole, merocyanine 540, resorufin, rose bengal, and 2,4-diphenyl-3(2H)-furanone).

A chemiluminescer involves a compound that becomes electronically excited by a chemical reaction and may then emit light which serves as the detectable signal or donates energy to a fluorescent acceptor. A diverse number of families of compounds have been found to provide chemiluminescence under a variety of conditions. One family of compounds is 2,3-dihydro-1,-4-phthalazinedione. The most popular compound is luminol, which is the 5-amino compound. Other members of the family include the 5-amino-6,7,8-trimethoxy- and the dimethylamino[ca]benz analog. These compounds can be made to luminesce with alkaline hydrogen peroxide or calcium hypochlorite and base.

Another family of chemiluminescence compounds is the 2,4,5-triphenylimidazoles, with lophine as the common name for the parent product. Chemiluminescent analogs include para-dimethylamino and -methoxy substituents. Chemiluminescence may also be obtained with oxalates, usually oxalyl active esters (e.g. p-nitrophenyl) and a peroxide (e.g., hydrogen peroxide), under basic conditions. Alternatively, luciferins may be used in conjunction with luciferase or lucigenins.

Various radioisotopes can be used to label compounds for use as signaling agents. Known radioactive labels include tritium (³H), radioactive iodine (¹²⁵I), radioactive carbon (¹⁴C), radioactive phosphorous (³²P), radioactive sulphur (³⁵S), radioactive calcium (⁴⁵Ca), radioactive chromium (⁵¹Cr), radioactive ruthenium (¹⁰³Ru), radioactive iron (⁵⁹Fe), radioactive zinc (⁶⁵Zn), radioactive selenium (⁷⁵Se), or the like. Methods for labeling of compounds with radioactive labels are well known in the art.

Molecular Recognition Agents

In certain embodiments, a functionalized nanochannel or nanocap includes a highly specific molecular recognition agent (FIG. 2). A molecular recognition agent of the invention can be selected such that it interacts only with the target analyte species to be detected. Molecular recognition agents that may be used include, but are not limited to, antibodies (including polyclonal and monoclonal antibodies), recombinant antibodies, chimeric antibodies, antigens, recombinant antigens, chimeric antigens, carbohydrates, lectins, nucleotide sequences (including recombinant, and chimeric nucleotides), peptide sequences (including recombinant and chimeric peptide sequences), polymeric acids, polymeric bases, protein binders, peptide binders, chelating agents (such as a crown ether or cryptand), aptamers, biochemical or biological (such as cellular) ligands or receptors, DNA aptamers, RNA aptamers, synthetic receptors, or a combination thereof.

According to the subject invention, molecular recognition agents may be attached to any surface of a functionalized or non-functionalized nanochannel or nanocap. In certain embodiments, molecular recognition agents are covalently attached to functionalized or non-functionalized nanochannel(s) or nanocap(s) via functional groups introduced by functionalization of the surface.

Alternatively, molecular recognition agents may be covalently attached to a functionalized or non-functionalized nanochannel or nanocap via linker molecules. Molecular recognition agents may also be attached to the functionalized or non-functionalized nanochannel or nanocap by non-covalent linkage, for example by absorption via hydrophobic binding or Van der Waals forces, hydrogen bonding, acid/base interactions and electrostatic forces.

The molecular recognition agent of the present invention can be an antibody specific to a target analyte. An antibody has a recognized structure that includes an immunoglobulin heavy and light chain. The heavy and light chains include an N-terminal variable region (V) and a C-terminal constant region (C). The heavy chain variable region is often referred to as “V_{H}” and the light chain variable region is referred to as “V_{L}”. The V_{H} and V_{L} chains form a binding pocket that has been referred to as F(v). See generally Davis, 3: 537, Ann. Rev. of Immunology (1985); and Fundamental Immunology 3rd Ed., W. Paul Ed. Raven Press LTD. New York (1993). Such structures facilitate highly effective and specific binding of an antibody to a target analyte.

Alternatively, recombinant bispecific antibody (bsFv) molecules can be used as molecular recognition agents of the invention. In one embodiment, bsFv molecules that bind a T-cell protein termed “CD3” and a TAA are used as molecular recognition agents in accordance with the present invention.

With other embodiments of the present invention, the molecular recognition agent is in the form of an aptamer. The discovery of the SELEX™ (Systematic Evolution of Ligands by EXponential enrichment) methodology enabled the identification of aptamers that recognize molecules other than nucleic acids with high affinity and specificity (see, for example, Ellington and Szostak, “In vitro selection of RNA molecules that bind specific ligands,” Nature, 346:818-822 (1990); Gold et al., “Diversity of oligonucleotide functions,” Ann. Rev. Biochem., 64:763-797 (1995); Tuerk and Gold, “Systematic evolution of ligands by exponential enrichment—RNA ligands to bacteriophage-T4 DNA-polymerase,” Science, 249:505-510 (1990), all of which are herein incorporated in their entirety by reference). Aptamers have been selected to recognize a broad range of targets, including small organic molecules as well as large proteins (see Gold et al., supra.; Osborne and Ellington, “Nucleic acid selection and the challenge of combinatorial chemistry,” Chem. Rev., 97:349-370 (1997), which is herein incorporated by reference in its entirety).

In certain embodiments, aptamers derived from the SELEX methodology may be utilized as molecular recognition agents in the present invention. The SELEX methodology is based on the insight that nucleic acids have sufficient capacity for forming a variety of two- and three-dimensional structures and sufficient chemical versatility available within their monomers to act as ligands (from specific binding pairs) with virtually any chemical compound, whether monomeric or polymeric. Molecules of any size or composition can serve as target analytes. See also Jayasena, S., “Aptamers: An Emerging Class of Molecules That Rival Antibodies for Diagnostics,” Clinical Chemistry, 45:9, 1628-1650 (1999), which is herein incorporated by reference in its entirety.

Aptamers that can be used in the present invention include those described in U.S. Pat. No. 5,656,739 (hereinafter the '739 patent, which is herein incorporated by reference in its entirety). The '739 patent describes nucleic acids as particularly useful assembly templates because they can be selected to specifically bind nonoligonucleotide target molecules with high affinity (e.g., Tuerk and Gold (1990), supra), and because they can hybridize by complementary base pairing. Both forms of recognition can be programmably synthesized in a single molecule or hybridized into a single discrete structure.

Aptamers can be attached to proteins utilizing methods well known in the art (see Brody, E. N. and L. Gold, “Aptamers as therapeutic and diagnostic agents,” J Biotechnol, 74(1):5-13 (2000) and Brody, E. N. et al., “The use of aptamers in large arrays for molecular diagnostics,” Mol Diagn, 4(4):381-8 (1999), both of which are herein incorporated by reference in their entirety). Such photo-cross-linkable aptamers allow for the covalent attachment of aptamers to proteins. More importantly, such aptamer-linked proteins can then be immobilized on a surface of a nanochannel or nanocap.

For example, aptamer-linked proteins can be attached covalently to a nanochannel or nanocap, including attachment of the aptamer-linked protein by functionalization of the surface of the nanochannel or nanocap. Alternatively, aptamer-linked proteins can be covalently attached to a functionalized or non-functionalized nanochannel or nanocap surface via linker molecules. Non-covalent linkage provides another method for introducing aptamer-linked proteins to a functionalized or non-functionalized nanochannel or nanocap surface. For example, an aptamer-linked protein may be attached to a functionalized or non-functionalized nanochannel or nanocap surface by absorption via hydrophilic binding or Van der Waals forces, hydrogen bonding, acid/base interactions, and electrostatic forces.

Assay Means and Transduction

In operation, the subject invention comprises at least three steps: (a) preparation of a sample for which target analyte analysis is to be conducted; (b) exposure of a nanosensing structure of the invention to the sample; and (c) detection of a signal (or signaling agent) that indicates detection (and/or concentration) of target analyte in the sample. Exposure of a nanosensing structure to a sample includes presentation of the sample to any surface of a membrane comprising the nanochannel(s) of the invention. For example, a sample can be presented to a surface of a membrane that exhibits tip opening(s) or, alternatively, to a membrane surface that exhibits base opening(s).

This concept of translating the presence of a target analyte in or near the nanochannel(s) into a detectable signal is called transduction. The assay means preferrably transduces target analyte detection/quantification into a signal that is communicated to the user. According to the subject invention, the assay means used to detect the target analyte can be of any of the vast array of available transduction strategies demonstrated for other types of sensing systems. Further, the magnitude of the transduction signal can be used to determine the concentration of the target analyte(s) in the sample. Hence, this invention not only detects but also quantifies.

In one embodiment, the nanosensing structure of the present invention uses the membrane having the at least one nanochannel therein in a detection system wherein the membrane and/or the shape of the nanochannel(s) cause the nanochannel(s) to be blocked, either permanently or transiently, by the target analyte. An assay means is then used to detect a characteristic change (i.e., optical, electrical, biochemical, chemical, acoustic, etc. characteristic) produced in response to detection and/or quantification of a target analyte.

In accordance with the subject invention, characteristic changes can include, but are not limited to: optical changes (i.e., optical changes observed by means of light source such as fluorescence, chemiluminescence, or electrogenerated chemiluminescence changes)); electrical changes (i.e., change in electrical current through at least one nanochannel in a membrane); changes in fluid flow (either gas or liquid) through at least one nanochannel; change in signal agent (i.e., target analyte-induces an increase or decrease in the concentration of a particular signal agent such as hydronium ion (see change in pH)). The assay means for detecting such characteristic changes include, but are not limited to, fluorescence spectroscopy; UV-VIS absorption spectroscopy; Raman spectroscopy; Fourier transform infrared spectroscopy (FTIR); nuclear magnetic resonance (NMR); electrochemical methods such as amperometry or cyclic voltammetry (if the signaling agent is redox active), potentiometry (if the signaling agent is an ion), and radiometric methods (if the signaling agent is radioactive); and the like.

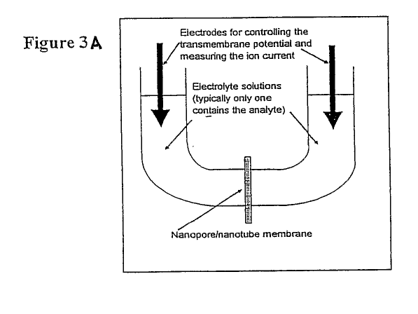

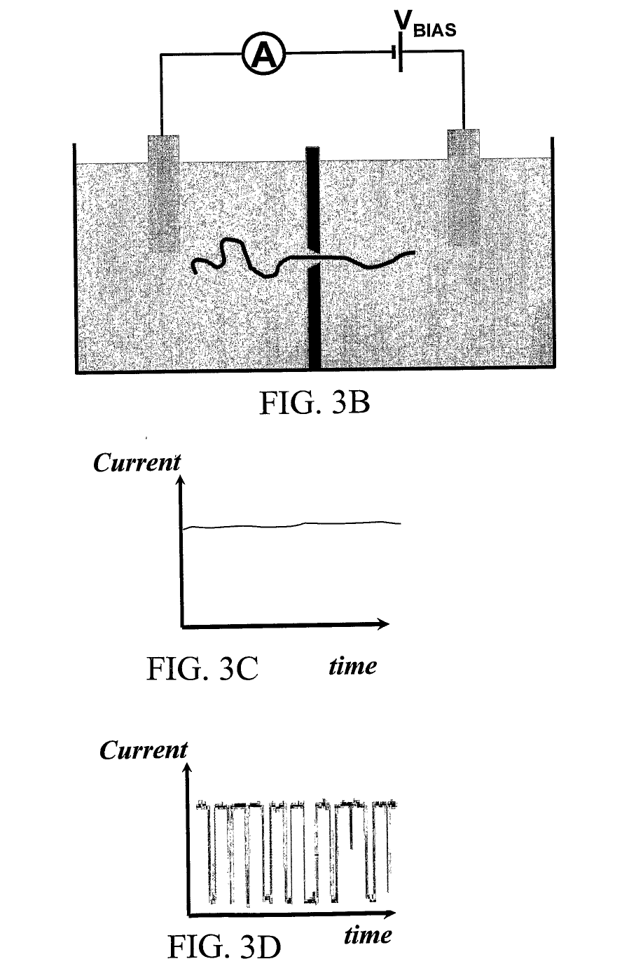

In one embodiment, the assay means involves various electronic instrumentation and/or circuits that enable application and detection of a voltage across a membrane of the invention, which comprises at least one nanochannel. In a related embodiment, as shown in FIGS. 3A and 3B, a membrane comprising at least one nanochannel is placed between two electrolyte solutions. In FIG. 3A, the nanochannel is functionalized. In FIG. 3B, the nanochannel is conical in shape. The analyte to be detected may be in one or both of the electrolyte solutions. The electrodes present in the electrolyte solutions are used to apply a transmembrane potential difference and to measure resulting transmembrane ion currents. The ion current(s) responds to the presence of the target analyte(s). Based upon the ion current response, the presence of the target analyte(s) may be detected.

As noted herein, the nanosensing structure may be designed such that any number of different responses may be selected to indicate the presence of the target analyte. In the particular example of ion-current detection, the responses to the analyte may include, but are not limited to, a decease or increase in the ion current, a total blockage of the ion current, a change in the current-voltage curve, a pattern of blockages (partial or total) of the ion current when measured as a function of time, a pattern of transient enhancements of the ion current when measured as a function of time, or a combination thereof. For example, as illustrated in FIGS. 3C (no target analyte) and 3D (target analyte present), each time an analyte causes the blockage of the nanochannel, the current flow through the nanochannel carried by ions from the solutions present on either side of the membrane is altered. The concentration of analyte is determined from the frequency of occurrence of the binding events. The identity of the analyte can be determined from the magnitude and duration of the current fluctuations.

While the above embodiments describe a detection system that is electrochemical in nature, other non-electrochemical transduction schemes may also be used in the present invention including, but not limited to, fluorescence, chemiluminescence and electrogenerated chemiluminescence.

In other embodiments, the assay means involves various known instrumentation that enable the flow of fluids across a membrane of the invention, which comprises at least one nanochannel. The flow of fluids can be generated by standard microfluidic methods such as hydrostatic pressure methods; hydrodynamic methods, electrokinetic methods, electroosmotic methods, hydromagnetic methods, acoustic methods, ultrasound methods, mechanical methods, electrical field induced methods, heat-induced methods, and other known methods. In certain embodiments, the flow of fluids across a membrane of the invention is generated osmotically or with a pump that generates positive pressure or negative pressure. Preferably, the assay means of such embodiments allow flow-rate control.

In further embodiments, the assay means involves various known techniques and/or instrumentation that enable the detection of signaling agents that are released from at least one nanochannel when exposed to a target analyte. For example, assay means of the invention for detecting signaling agents include, but are not limited to, gross examination (e.g., detection with the human eye/by observation); radiographic systems; and microscopic systems (e.g., light microscopy, transmission electron microscopy, and laser capture microscopy).

Analytes and Samples

The present invention provides useful nano-based sensing systems for the detection and/or quantification of various analytes. Specific analytes to be detected and/or measured in accordance with the present invention include, but are not limited to, toxins, organic compounds, proteins, peptides, microorganisms, amino acids, carbohydrates, nucleic acids, hormones, steroids, vitamins, drugs (including those administered for therapeutic purposes as well as those administered for illicit purposes), virus particles and metabolites of or antibodies to any of the aforementioned substances. For example, such analytes include, but are not intended to be limited to, spores, pollen, dust particles, ferritin; creatinine kinase MIB (CK-MIB); digoxin; phenyloin; phenobarbitol; carbamazepine; vancomycin; gentamycin; theophylline; valproic acid; quinidine; leutinizing hormone (LH); follicle stimulating hormone (FSH); estradiol, progesterone; IgE antibodies; vitamin B2 micro-globulin; glycated hemoglobin (Gly. Hb); cortisol; digitoxin; N-acetylprocainamide (NAPA); procainamide; antibodies to rubella, such as rubella-IgG and rubella-IgM; antibodies to toxoplasmosis, such as toxoplasmosis IgG (Toxo-IgG) and toxoplasmosis IgM (Toxo-IgM); testosterone; salicylates; acetaminophen; hepatitis B virus surface antigen (HBsAg); antibodies to hepatitis B core antigen, such as anti-hepatitis B core antigen IgG and IgM (Anti-HBC); human immune deficiency virus 1 and 2 (HTLV); hepatitis B e antigen (HBeAg); antibodies to hepatitis B e antigen (Anti-Hbe); thyroid stimulating hormone (TSH); thyroxine (T4); total triiodothyronin (Total T3); free triiodiothyronin (Free T3); carcinoembryoic antigen (CEA); and alpha fetal protein (AF); and drugs of abuse and controlled substances, including but not intended to be limited to, amphetamine; methamphetamine; barbituates such as amobarbital, seobarbital, pentobarbital, phenobarbital, and barbital; benzodiazepines such as librium and valium; cannabinoids such as hashish and marijuana; cocaine; fetanyl; LSD; methapualone; opiates such as heroin, morphine, codine, hydromorphone, hydrocodone, methadone, oxycodone, oxymorphone and opium; phencyclidine; and propoxyhene. The term analyte also includes any antigenic substances, haptens, antibodies, proteins (such as toxins, e.g., Ricin), DNA, RNA, macromolecules and combinations thereof.

A sample of the invention is a material suspected of containing a target analyte. The sample of the invention can be presented in any form suitable for analysis including a solid, liquid, or gas phase. The sample of the invention can be used directly as obtained from the source or following a pre-treatment to modify the character of the sample. For example, the test sample can be can be derived from any biological source such as, but not limited to, exhaled breath, whole blood, blood plasma, saliva, ocular lens fluid, cerebral spinal fluid, semen, sweat, mucous, urine, milk, ascites fluid, mucous, synovial fluid, lymph fluid, meningal fluid, peritonaeal fluid, amniotic fluid, glandular fluid, sputum, feces, fermentation broths, cell cultures, chemical reaction mixtures, and the like. In addition to biological or physiological fluids recited herein, other liquid samples can be used such as water, food products, aerosol collectors, and the like for the performance of environmental or food production assays.

A sample of the invention can be pretreated prior to analysis to disperse a target analyte into a preferred media (i.e., solution, aerosol, gas phase, etc.). Methods of treatment can involve filtration, distillation, concentration, inactivation of interfering components, and the addition of reagents. For example, a sample can include experimentally separated fractions of solutions or mixtures containing homogenized solid sample material, such as feces, tissues, and biopsy samples. In certain embodiments, a solid test sample is modified to form a liquid medium or to release a target analyte. In a related embodiment, a swab is wiped across a surface suspected of having a target bioterror analyte on it, and this analyte is rinsed off of the swab into a solution, which would then be analyzed for the target bioterror analyte using a nano-based system of the invention.

Following are examples that illustrate the versatility of the nanosensing structures of the present invention as well as procedures for practicing the invention. These examples should not be construed as limiting in any manner of the overall scope of the present invention. All percentages are by weight and all solvent mixture proportions are by volume unless otherwise noted.

### EXAMPLE 1

### pH-Based Sensing System

In one embodiment, a nano-based sensing system of the present invention detects the presence of a target analyte via an assay means that observes a change in pH. The assay means of the invention translates a change in pH into a change in the current-voltage curve, which communicates to the user the presence of the target analyte. Such sensing system is particularly advantageous for use in measuring different concentrations of target analytes in any given sample medium.

In this example, the membrane is a polyethylene terephthalate (PET) film within which a single conical nanochannel was functionalized with a gold lining. The large (base) diameter opening of the nanochannel was about 0.6 μm and the small (tip) diameter opening was about 30 nm. An electroless plating method was used to deposit a corresponding the gold lining within the PET conical nanochannel. Because the gold (or Au) lining was so thin, the large diameter opening of the gold-lined nanochannel remained about 0.6 μm. The mouth diameter of the lined nanochannel was controlled by varying the electroless plating time and measured using a simple electrochemical method. A pH-response molecule (such as 2-mercaptopropionic acid, MPA) was attached to the gold surface via an —SH linker. According to the subject invention, a pH-response molecule can be attached to any gold surface of a nanochannel, including the inside surface of the nanochannel, as well as the front and back surfaces of the membrane in which the nanochannel is located.

Attachment of a thiol (—SH) linker group that spontaneously adsorbs to gold functionalizes the nanochannel surface for linkage with a molecular recognition agent (such as, in this example, a pH-response molecule). A molecular recognition agent can be attached to the gold surface of a gold-lined nanochannel by covalent bonds, such as disulphide bonds via an-SH linker group. An enormous array of thiol-containing molecules are commercially available for use in functionalizing the gold surface of a nanotube to present an activated disulfide bond for linkage with a molecular recognition agent (see Example 2 for another example of a thiol-containing molecule that attaches to a gold nanotube).

As described herein, functionalization of nanochannel surface can be performed using methods well known to the skilled artisan (see, for example, U.S. Patent Application Publication No. 2004/0076681, the disclosure of which is herein incorporated by reference). For example, gold-lined nanochannel surfaces can be functionalized while the lining is embedded in the nanochannel(s) of a membrane. This ability to very conveniently chemically and biochemical functionalize gold surfaces make gold-lined nanochannels a particularly beneficial embodiment of the present invention. However, as discussed herein, other lining materials may also be conveniently used to functionalize the nanochannel(s) of the invention.

The membrane with the gold-lined nanochannel was mounted between the two halves of a conductivity cell, and each half-cell was filled with about 1.7 mL of a 1M phosphate buffer solution that was also 1 M in KCl. A Ag/AgCl electrode was inserted into each half-cell solution, and an Axopatch 200B (Axon Instruments, Sunnyvale, Calif.) was used to apply the desired transmembrane potential and to measure the resulting ion current flowing through the nanochannel.

In one embodiment, to provide a control and example for comparison, the measurement procedure was as follows: (a) obtain a current voltage (I-V) curve before exposure to target analyte; (b) replace the solution facing the mouth of the nanochannel with an electrolyte solution of an analyte that does not affect the nanochannel (i.e., analyte that does not bind to the molecular recognition agent) and obtain a second I-V curve; and (c) replace the solution of step (b) with a solution of a target analyte that does affect the nanochannel and obtain the I-V curve.

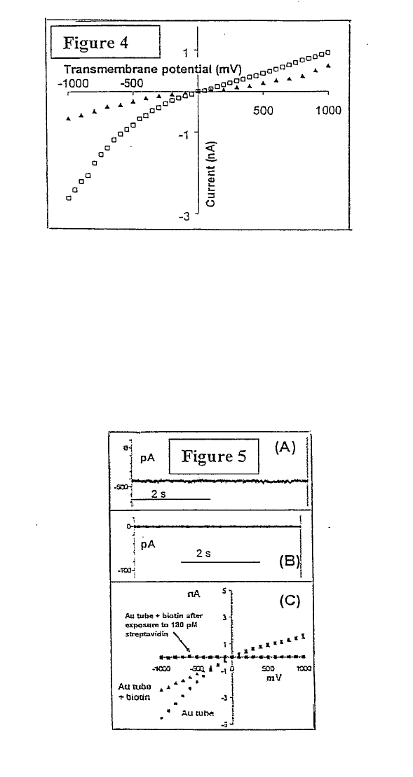

I-V curves for gold-lined nanochannels modified with 2-mercaptopropionic acid (MPA) were recorded in Karl Fischer (KF) electrolyte at different pH values, which are shown in FIG. 4. At the lower pH the I-V curve is linear, while at the higher pH the I-V curve is assymetrical. The assymetry observed at high pH results from the deprotonation of the MPA to render the gold-lined nanochannel walls (and membrane surfaces) negatively charged. FIG. 4 demonstrates that a pH-based nanosensing system may be prepared via the technology described in this invention.

### EXAMPLE 2

### Biocompound-Based Sensing System (Biotin)

In this example, the present invention uses a membrane with a gold-lined nanochannel and at least one molecular recognition agent attached to the gold-lined nanochannel walls, where the molecular recognition agent responds selectively to a protein. The gold-lined nanochannel had a small (tip) diameter opening of about 5 nm and a large (base) diameter opening of about 0.6 μm. The molecular recognition agent attached to the gold lining was the biomolecule biotin. Attachment to the gold lining was accomplished using a commercially available biotin molecule having a thiol functionality. Biotin binds with high specificity and strength to the protein Streptavidin (SA). Hence, in this example SA is the target analyte.

The membrane with the gold-lined nanochannel functionalized with biotin was mounted between the two halves of a conductivity cell, and each half-cell was filled with about 1.7 mL of a 1M phosphate buffer solution (pH=4.5 for IgG and ricin, pH=9 for SA) that was also 1 M in KCl. A Ag/AgCl electrode was inserted into each half-cell solution, and an Axopatch 200B (Axon Instruments, Sunnyvale, Calif.) was used to apply the desired transmembrane potential and to measure the resulting ion current flowing through the nanochannel.

The measurement procedure was as follows: (a) obtain a current voltage (I-V) curve before exposure to SA protein; (b) replace the solution facing the mouth of the nanochannel with an electrolyte solution of a protein that does not affect the nanochannel (i.e., binds to the molecular recognition agent) and obtain a second I-V curve; and (c) replace the solution of step (b) with a solution of a SA protein that does affect the nanochannel and obtain the I-V curve.

FIG. 5A shows a current vs. time trace for the conical gold-lined nanochannel having the biotin ligand attached to the gold surfaces prior to exposure to the target analyte protein SA. A constant transmembrane ion-current is observed. FIG. 5B shows the current vs. time trace after exposure to a solution that was 180 pM in the target analyte protein SA. The binding of the protein to the biotin at the tip opening of the nanochannel caused the tip opening to be blocked, such that the ion current was substantially zero, thereby indicating the presence of the analyte.

FIG. 5C shows analogous data in the form current voltage curves. The current voltage curves for the gold-lined nanochannel before and after attachment of the biotin are shown. Note that the biotin partially blocks the tip opening of the gold-lined nanochannel so that the current decreases after biotin adsorption. This is in itself an important result because it shows that this new sensing strategy may be used to sense small molecules, in this case the molecule biotin (molecular weight=244). After exposure to the analyte SA, again, the current (at any applied transmembrane potential) was zero.

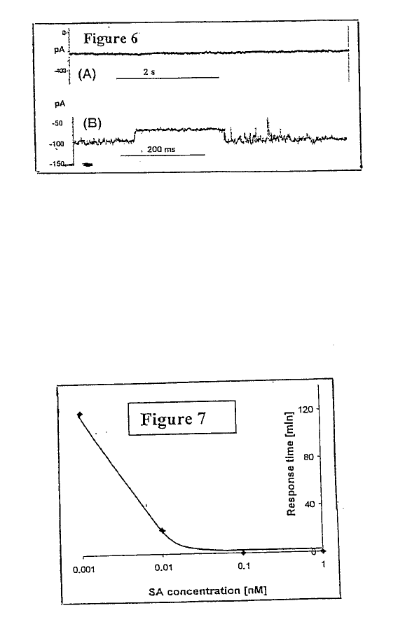

The blockage of the nanochannel tip opening has been interpreted as resulting from the binding of the analyte SA to the nanotube bound ligand biotin. However, it was possible that the blockage was simply caused by non-specific adsorption of the protein to the nanotube walls. FIG. 6 shows a control experiment that proved that this was not the case. FIG. 6 shows current vs. time traces for a conical gold-lined nanochannel (where the nanochannel is functionalized with biotin) sensing system of the invention before and after exposure to a much higher concentration (100 nM) of the protein lysozyme. Lysozyme does not bind to the ligand biotin, and for this reason, permanent blockage of the nanotube tip opening was not observed, and ion-current always flowed through the nanotube. Such data proves that the biotin-containing gold-lined nanochannel was a specific sensor for the protein SA (FIG. 5) and not a non-specific protein detector. The lower current in FIG. 6B resulted because the transmembrane potential in this case (−50 mV) was lower than in FIG. 6A (−200 mV).

FIG. 6 illustrates another aspect of the present invention. While the biotin-based nanochannel sensor of the invention specifically recognized SA, it was not blind to lysozyme. It detected lysozyme as transient and partial blockage of the ion current. These transient-blockage events are associated with passage of the protein molecule through the nanochannel tip opening. This is an example of the well-known sensing method called stochastic sensing, which is typically practiced with a biological protein nanopore embedded within a lipid bilayer membrane.

As will be discussed in greater detail in a later example, stochastic sensing is, in fact, not non-specific because the duration and magnitude of the current blockade (e.g., FIG. 6B) is related to the size, shape, charge, and chemistry of the analyte species passing through the nanopore. Such stochastic sensing paradigm is another important way the nanosensing structures that are the subject of this patent may be used.

The results in FIGS. 5 and 6 illustrate that the nanotube sensor with an attached ligand, according to one embodiment of the present invention, may selectively detect an analyte species that binds to the ligand. However, the response is in an “on/off” (current flow/no current flow) and/or “yes/no” (analyte present/analyte not present) fashion. For example, detection of an ion current flows when an analyte is not present (FIG. 5A, on/no signal) and no detectable current flow when the analyte binds to the molecular recognition agent (FIG. 5B, off/yes signal). This is an extremely useful function (for example in sensing whether an environment has been exposed (yes) or has not been exposed (no) to a bio-warfare agent).

In another embodiment of the invention, the subject sensing platform is used to identify the presence of a target analyte as well as obtain the analyte concentration. In a related embodiment, detection and communication of target analyte concentration is accomplished using the sensor of this Example 2 by monitoring the time required for the analyte to shut off the ion current.

FIG. 7 illustrates this point as a calibration curve of time required for the analyte SA to shut off the ion-current in the biotin-functionalized nanotube vs. SA concentration. As would be expected on the basis of mass-transport from the analyte solution to the nanochannel tip opening, the time required to shut off the ion current is inversely proportional to the analyte concentration. While the times for the lowest concentration are long in FIG. 7, they may easily be shortened by applying a larger transmembrane potential to electrophoretically drive the analyte protein to the nanochannel tip opening. This aspect has been shown by studying the fluxes of proteins through such nanochannels as a function of applied transmembrane potential. As would be expected, protein flux increased with applied transmembrane potential.

The concept of using the time required to block the nanochannel opening by the analyte is only one of many possible ways to quantify target analyte concentration. There are numerous other methods to quantify target analyte concentration using the nanochannel sensing strategy of the subject invention. These include any one or combination of the following methods:

A. Using a pH sensing device discussed in Example 1 above, where the analyte concentration (in the case of Example 1, hydronium ion concentration) changes the shape of the current-voltage curve for a single nanochannel or multiple nanochannels on a membrane substrate.

B. Using a membrane that contains many nanochannels as the sensing element where the number of nanochannels that get shut off (detection of no ion current flow) is determined by the concentration of the target analyte in the sample medium. Accordingly, the magnitude of the measured current is inversely proportional to the analyte concentration.

C. Using a membrane that contains many nanochannels as the sensing element where the tip openings of a multi-nanochannel membrane are initially blocked by nanoparticles. Because of the blockage of the nanochannel openings, ion-current cannot flow through the nanochannels in the membrane. When a target analyte is present, the analyte displaces the blocking nanoparticles to enable ion current flow through the unblocked nanochannel openings. With this method, the detectable, measured current is directly proportional to analyte concentration.

D. Using a membrane that contains a single nanochannel where the tip opening of the nanochannel is initially blocked by a nanoparticle. Because of the blockage of the nanochannel opening, ion-current cannot flow through the nanochannel in the membrane. When a target analyte is present, the target analyte displaces the blocking nanoparticle to enable ion current flow (or “turn the current on”) through the unblocked nanochannel opening. The time required to turn the current on is related to analyte concentration.

With the sensing system of methods C or D above, a means for attaching the nanoparticle to the tip opening of the nanochannel(s) is required. Further, a means for the analyte to displace the blocking nanoparticle(s) is required. In one embodiment, these requirements are addressed by attaching a molecular recognition agent (such as, in this example, biotin) to a surface of the nanochannel, preferably on surface near the tip opening of the nanochannel. A nanoparticle is then prepared that has the analyte to be detected attached (such as, in this example, SA) to the nanoparticle surface. The diameter of this nanoparticle is slightly larger than the opening tip diameter of the nanochannel.

The nanochannel surface that contains the molecular binding agent is then exposed to the analyte-tagged nanoparticle. The analyte on the nanoparticle binds to the molecular recognition agent, effectively blocking the opening of the nanochannel. When the membrane is then exposed to a solution containing the target analyte, the analyte in the solution competes with the analyte attached to the nanoparticle. As a result of this competition, the analyte from the solution displaces the nanoparticle by binding to the molecular recognition agent(s) that held the nanoparticle in place. Since the analyte displaces the nanoparticle to unblock the nanochannel opening, the transmembrane ion current gets switches from “off” (nanoparticle blocking the tip opening) to “on” (nanoparticle displaced).

F. Using a membrane that contains a single nanochannel or multiple nanochannels as the sensing element where the tip opening(s) of the nanochannel(s) is initially blocked by detectable nanoparticle(s). Either the nanoparticle itself is detectable (for example, the nanoparticle is a fluorescent compound) or a detectable compound is affixed to the nanoparticle (for example, a fluorescent compound is attached to the nanoparticle). For example, the nanocaps would emit a detectable, fluorescence intensity when blocking the opening(s) of the nanochannel(s). When the blocking nanocaps are displaced by a target analyte, the fluorescence intensity from the sensing membrane surface would decline, indirectly signaling the presence and/or concentration of the target analyte. For example, a measurement of rate in decline in fluorescence intensity corresponds to concentration of target analyte in sample medium.

G. Using a membrane that contains a single nanochannel or multiple nanochannels as the sensing element where the tip opening(s) of the nanochannel(s) is initially blocked by detectable nanoparticle(s). An easily detectable species (e.g., a fluorophor, dye, quantum dot, metal nanoparticle, and the like) is confined within the nanochannel as a result of nanocap blockage of nanochannel opening(s). A target analyte causes the detachment of the nanocap to release the detectable species from the nanochannel(s). The concentration of target analyte in the sample medium is directly proportional to the concentration of detectable species released from the nanochannels.

### EXAMPLE 3

### Biocompound-Based Sensing System (Protein G)

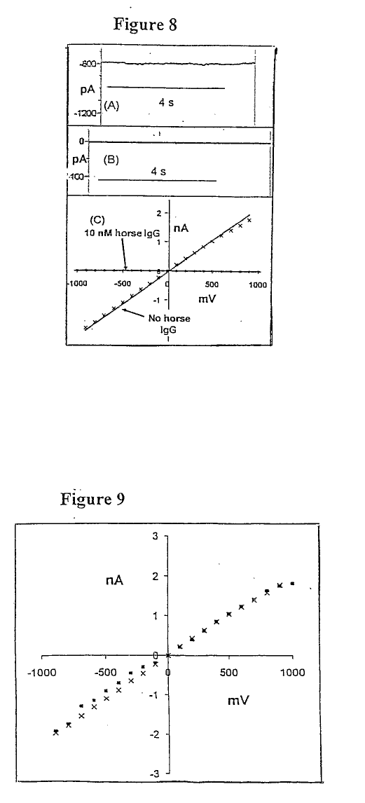

According to one embodiment of the invention, the sensor system comprises a nanochannel lined with gold and having a protein ligand (molecular recognition agent) attached to the gold lining of the nanochannel. The ligand was selected such that it responded selectively to a protein that binds to the ligand. In Example 2 above, the ligand biotin that selectively binds to the analyte SA was a small molecule. As illustrated in FIG. 8, the ligand may also be a protein. In this Example 3, the ligand was a protein, protein G. The particular protein G used in this example binds strongly to immunoglobulin G (IgG, an antibody) obtained from horse blood. Accordingly, the target analyte for the sensor system of Example 3 was the specific protein horse IgG.

The gold-lined nanochannel had two openings at the tip and base, a small-diameter tip opening of 4 nm and a large-diameter base opening of 0.6 μm. The gold-lined nanochannel is preferably conical in shape. The ligand, protein G, was immobilized to the walls of the gold-lined nanochannel by first attaching the thiol-containing biotin molecule used in Example 2. The biotinylated nanochannel was then exposed to the protein streptavidin, SA, to attach SA to the nanochannel walls. SA has four biotin binding sites per protein molecule, and only one of these sites is used to attach the SA to the nanochannel walls. The other three sites may bind additional biotin molecules, and these available binding sites were used to attach a commercially available biotin-labeled protein G to the nanochannel walls. Preferably, the ligand (protein G) was immobilized around the tip opening of the nanochannel to ensure blockage of the opening when binding to a target analyte.

This bio-functionalization strategy illustrates how easy it is to functionalize the gold-lined nanochannels described herein. However, again, other materials used in the manufacture of nanochannels in accordance with the subject invention may just as easily be functionalized.

FIG. 8A shows a current vs. time trace for the conical, gold-lined nanochannel having protein G ligand attached to the gold surfaces prior to exposure to the target analyte, protein horse IgG. A constant transmembrane ion-current is observed prior to exposure to the target analyte. FIG. 8B shows the current vs. time trace after exposure to a solution that contained 10 nM of the target analyte, protein horse IgG. The binding of the protein horse IgG to the protein G at the tip opening of the nanochannel caused the opening to be blocked, and therefore disrupt the flow of ion current (ion current is preferably zero) to provide indication that the target analyte was present in the solution. FIG. 8C shows analogous data in the form of I-V curves. After exposure to the analyte, again, the current (at any applied transmembrane potential) is zero.

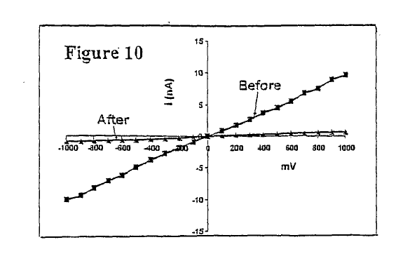

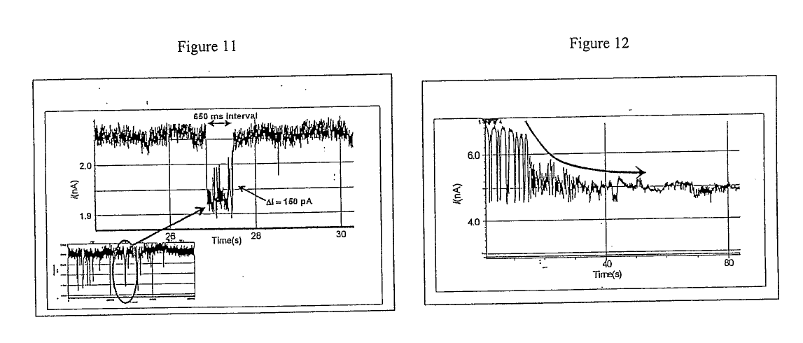

As with Example 2 (where SA was the target analyte), the blockage of ion current as illustrated in FIG. 8 was due to the specific binding of the analyte to the ligand and not due to non-specific adsorption of the protein to the nanochannel gold-lined walls. FIG. 9 confirms that the opening blockage was due to the specific ligand/analyte interaction. FIG. 9 shows I-V curves for a conical, gold-lined nanochannel sensor having attached thereto protein G before and after exposure to 10 nM IgG from cat blood. The cat IgG did not bind to the protein G attached to the nanochannel walls and, as a result, there was no change in the I-V curve after exposure to cat IgG. These results very clearly establish that a protein G functionalized sensor, as disclosed herein, is highly specific for the analyte protein horse IgG as opposed to protein IgG from other species (such as cat IgG).

### EXAMPLE 4

### Particle Sensing System

In Example 4, the ability to detect the presence of target analytes using a sensor system of the invention is demonstrated. According to the subject invention, particles such as biological cells, spores and virus may be regarded as target analytes. These particles have specific biochemical labels (e.g., specific proteins or sugars) on their surfaces. When a molecular recognition agent having the ability to bind to a biochemical label (of the particle) is attached to a surface of a nanochannel in a membrane substrate, a sensor system is provided to detect the particle.

In one embodiment, the molecular recognition agent is attached to a surface near the opening of the nanochannel. Detection of target analyte presence entails blockage of the opening of the nanochannel via selective binding of the particle to the molecular recognition agent. One method of transducing nanochannel opening blockage involves monitoring a transmembrane ion current. In a related embodiment, the opening diameter of the nanochannel is manipulated. For example, both the tip opening diameter and the diameter of a substantial portion of the nanochannel are controlled to serve the user's needs, including limiting the diameter from about <1 nm to >10's of microns.

To explore the particle sensing model of Example 4, commercially available 10 nm diameter colloidal gold-lined nanoparticles with the protein streptavidin (SA) attached to the particle surface were provided as target analytes. The sensor of the invention comprised a gold-lined nanochannel having a small-diameter tip opening of 40 nm and a large-diameter of 5 μm substantially throughout the remainder of the nanochannel. The biotin-thiol discussed in Example 2 was attached to the gold-lined surface of the nanochannel, preferably near the tip opening of the nanochannel. As with Example 3, binding of SA on the target analyte to biotin blocked the tip opening of the nanochannel.

FIG. 10 illustrates the particle sensing concept of this Example, in which ion-current flow provides a signaling means for analyte detection. The transmembrane ion-current, after binding of the SA/target analyte to the biotin/molecular recognition agent, was substantially lower than the ion current prior to introduction of target analyte. The ion-current did not drop to zero because it was impossible for 10 nm-diameter analyte nanoparticles to completely block a 40 nm nanochannel tip opening. As is clearly illustrated in FIG. 10, complete blockage was not essential for detecting the presence of the nanoparticle/target analyte. If complete blockage was desired, a nanochannel with a smaller diameter tip opening could be used. Finally, analogous colloidal gold particles that did not have SA attached to their surfaces did not result in a permanent drop in the transmembrane ion current.

### EXAMPLE 5

### Biocompound Sensing System (DNA)

In Example 5, the ability of the sensing device to detect DNA was shown. A gold-lined nanochannel with a molecular recognition agent (single-stranded DNA; ssDNA) attached to the nanochannel walls was used, wherein the single-stranded DNA is selective for complementary single-stranded DNA (target analyte). Functionalization of the nanochannel walls with the molecular recognition agent-ssDNA was easily accomplished using commercially available DNAs that have a terminal thiol.

In this particular Example 5, the molecular recognition agent-ssDNA was 5′ SH—(CH₂)₆—CGC GAG AAG TTA CAT GAC CTG TAG ACG ATC 3′ (C=cytosine, T=thymine, G=guanine, A=adenine). The target analyte was the complementary 30-mer 5′ GCG CTC TTC AAT GTA CTG GAC ATC TGC TAG 3′.

To prove that the subject sensor did, indeed, specifically recognize the target analyte DNA, the response to a DNA chain that was non-complementary to the ligand-DNA was also investigated. This non-complementary DNA was a DNA chain consisting of 32 Ts.

The DNA sensor of Example 5 comprised of a gold-lined nanochannel having a small, tip opening diameter of 40 nm and a large-diameter opening substantially throughout the remainder of the nanochannel of 5 μm. FIG. 11 shows current vs. time traces for this sensor after exposure to a solution that contained 5 nM of the non-complementary DNA molecule. As has been discussed previously (FIG. 6B), because this non-target analyte molecule did not bind to the molecular recognition agent-ssDNA attached to the nanochannel walls, transient current blockades were observed, where these blocks were associated with passage of this non-target analyte DNA through the tip opening of the conical nanochannel.

FIG. 12 shows analogous data after exposure to a solution that contained 5 nM of the target analyte DNA that was complementary to the molecular recognition agent-ssDNA. Transient current blockades were initially observed; however, this target analyte DNA bound to the molecular recognition agent-ssDNA, and partially occluded the nanochannel tip opening. As a result, the current was ultimately and permanently decreased to a lower level. Again, the current for this sensor was not completely shut off because the diameter of the tip opening of the nanochannel was greater than the diameter for the target analyte. While not essential, if complete blockage is desired, a nanochannel with a smaller tip opening diameter could be used.

### EXAMPLE 6

### Stochastic Sensing System

In Example 6, the present invention is shown to be useful in stochastic sensing (SS). SS is an important biosensing technology that has been demonstrated to be applicable to a large number of different analytes. According to the subject invention, SS may be used in conjunction with the nanosensing systems of the invention. For example, SS may be done with a protein channel embedded at an opening of a nanochannel or within a lipid bi-layer membrane covering the opening to the nanochannel.

In SS, the analyte species is translocated through the protein channel and to the nanochannel, and when in the nanochannel, it binds to a surface of the nanochannel (or blocks the nanochannel passage) to partially/wholly blocks the pathway of ionic conduction through the nanochannel. Hence, the signal in SS is a series of current-block events. The frequency of these events is inversely related to the analyte concentration, and the magnitude and duration of these events provide the chemical identity of the analyte. SS may be done with a molecular recognition agents attached to the nanochannel, or an uninterrupted nanochannel may be used.

According to the subject invention, synthetic nanochannels can be used to perform SS (FIGS. 6B, 11, 12). The ability to do SS in synthetic nanochannel systems, and the ability to produce a practical SS technology based on these systems, can be performed in different embodiments of the present invention, as described herein.

### EXAMPLE 7

### Stochastic Sensing with α-hemolysin Protein Channels at the Opening of a Nanochannel

α-hemolysin (αHL) is an exotoxin secreted by Staphylococcus aureus. The (αHL protein is a 293-amino acid chain, and seven chains self-assemble on the lipid bilayer membrane to form the αHL channel. The αHL protein channel can be pre-assembled in solution and then inserted into a lipid bilayer membrane (see, for example, Braha, O. et al., Chem. Biol., 4:497 (1997); Cheley, S. et al., Chem. Biol., 9:829-838 (2002); Shin, S. et al., Chem. Int. Ed., 41:3707 (2002); Gu, L. et al., Nature, 398:686 (1999); Sanchez-Quesada, J. et al., J. Am. Chem. Soc., 122:11758 (2000); Gu, L. et al., Science, 291:636 (2001); Movileanu, L. et al., Nature Biotechnology, 18:1091 (2000); Howorka, S. et al., Proc. Natl. Acad. Sci. USA, 98:12996 (2001); Howorka, S. et al., Nature Biotechnology, 19:636 (2001)).

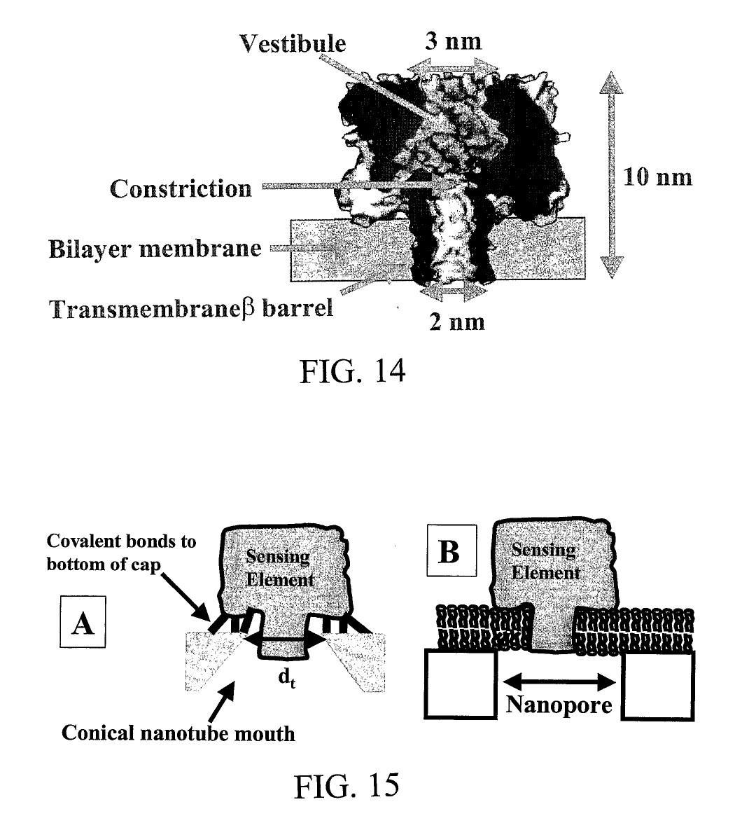

In addition, the αHL protein can be chemically and genetically engineered to build molecule-specific binding sites within the channel. The channel can then be assembled from seven of these engineered proteins (homomer) or from a mix of engineered and wild type chains (heteromer). The crystal structure of the channel has been determined (see Song, L. et al., Science, 274:1859 (1996)). The αHL protein channel is shaped like a mushroom (FIG. 14) to form a cap over a nanochannel, where the αHL protein channel has a stem that is ˜2 nm in diameter and a cap that is ˜10 nm in diameter.

In one nanosensing device of the invention, the molecular recognition agents (the αHL protein channel) are immobilized within the mouths of nanochannels (FIG. 15A) that run through a 10 μm-thick support membrane. In a related nanosensing device of the invention, the molecular recognition agents (the αHL protein channel) are immobilized within a mechanically rugged supported lipid bilayer membrane covering the surface and openings of nanochannels in a support membrane (FIG. 15B).

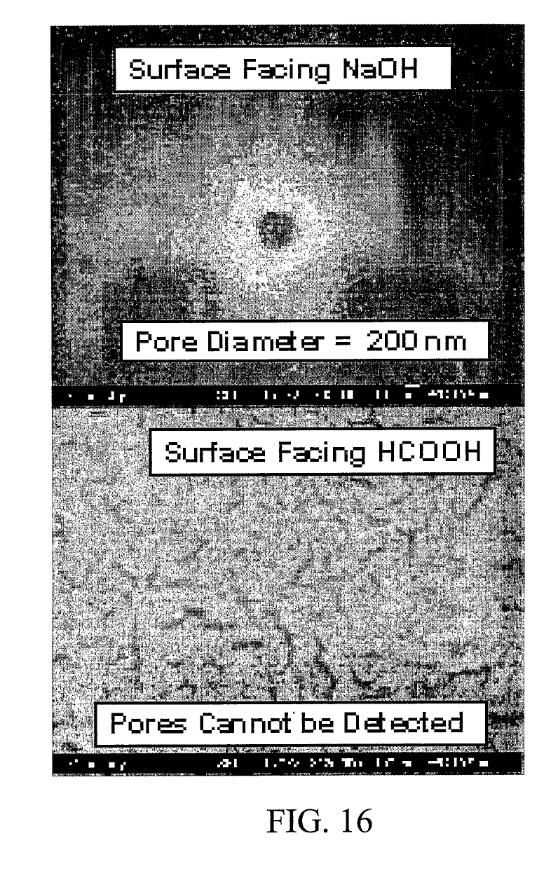

To prepare the nanochannel(s) of the invention, a track-etch technique was utilized. An anisotropic etch/stop bath is used in which the standard etch solution (6 M NaOH) is placed on the “etch” side of a membrane and a “stop” solution (2 M KCl plus 2 M formic acid) is placed on the “stop” side of the membrane. The formic acid serves to neutralize hydroxide anions that diffuse through the nascent pore from the etch side. FIG. 16 shows scanning electron micrographs of the surfaces of an ultralow pore density track-etched membrane that had been etched in this anisotropic stop/etch bath. The upper micrograph shows that at the surface exposed to the etch solution, the damage track has been etched into a 200 nm-diameter pore. The lower micrograph shows that the pores at the surface exposed to the stop solution are too small to be seen with this electron microscope (dₜ<3 nm).

As illustrated in FIG. 15, there are two approaches for immobilization of the protein-channel sensing element at the surface of the nanochannel in a support membrane. The first method (FIG. 15B) entails forming a mechanically rugged supported lipid bilayer membrane (SLBM) (Cremer, P. and Yang, T., J. Am. Chem. Soc., 121:8130 (1999); E. Sackmann, Science, 271:43 (1996); and Groves, J. et al., Science, 275:651 (1997)) across the surface such that it bridges the mouths/openings of the nanochannel(s), and then immobilizing the molecular recognition agents (e.g., αprotein channels within this SLBM (FIG. 5B). The advantages of this approach are: (a) the molecular recognition agents (e.g., α-hemolysin protein channels) are designed through evolution to spontaneously insert into lipid bilayer membranes, and (b) because this chemistry is completely analogous to conventional black lipid membrane technology (see, for example, Braha, O. et al., Chem. Biol., 4:497 (1997); Cheley, S. et al., Chem. Biol., 9:829-838 (2002); Shin, S. et al., Chem. Int. Ed., 41:3707 (2002); Gu, L. et al., Nature, 398:686 (1999); Sanchez-Quesada, J. et al., J. Am. Chem. Soc., 122:11758 (2000); Gu, L. et al., Science, 291:636 (2001); Movileanu, L. et al., Nature Biotechnology, 18:1091 (2000); Howorka, S. et al., Proc. Natl. Acad. Sci. USA, 98:12996 (2001); Howorka, S. et al., Nature Biotechnology, 19:636 (2001)), no additional chemistry is required to form a seal between the molecular recognition agent and the bilayer membrane. Methods for forming SLBM across the surfaces of nanochannels within membranes as well as methods for using SLBM to bridge the nanochannels at the surface of the membrane are known (see, for example, Hennesthal C. and Steinem, C., J. Am. Chem. Soc., 122:8085 (2000)).

The Langmuir-Blodgett (LB) method (see Tamm, L. K. and McConnell, H. M., Biophysical J., 47:105 (1985)) and a vesicle fusion method (see Kalb, E. et al., Biochim. Biophys. Acta, 1103:307 (1992)) were used to coat the surfaces of nanochannel support membranes with phospholipid SLBMs. In the LB method, the nanochannel support membrane is drawn vertically though an air/water interface containing a lipid monolayer. On hydrophilic surfaces, this process transfers a single layer of lipids onto the substrate with the hydrophobic tails pointing into the air and the hydrophilic headgroups facing the surface (see Tamm supra.). The upper lipid layer is then formed by horizontally dipping the substrate back through the interface. Where the nanochannel support membrane is a polycarbonate surface, the membrane is exposed to SO₃ gas to attach hydrophilic —SO₃H groups to the membrane surface. The skilled artisan would readily understand that SLBMs can be formed at such sulfonated polycarbonate surfaces.

In the vesicle fusion method, small unilamellar vesicles are formed by extrusion technique, and a solution of these vesicles is applied to the nanochannel support membrane surface. Again, the surface (if a polycarbonate surface) will be sulfonated so as to increase hydrophilicity. This method has already been used to coat the surface of a nanopore membrane such that the pore mouths are bridged by the SLBM (see, for example, Hennesthal C. and Steinem, C., J. Am. Chem. Soc., 122:8085 (2000). Furthermore, Fertig and coworkers have used this method to from a SLBM across a single nanopore in a track-etched quartz membrane (Fertig, N. et al., J. Chem. Phys. Rev. E, 64:040901(R) (2001)). Ion channels were immobilized channels into the track-etched quartz membranes and ion current measurements were made through the immobilized ion channel.

To ensure that the molecular recognition agents are immobilized within such SLBMs and that they do not diffuse in two dimensions across the membrane surface within the SLBM, a protein photolithographic method is utilized that allows impermeable “protein corrals” to be drawn in SLBMs. Specifically, such protein corrals are drawn into the portions of the SLBM above the nanochannel openings/mouths and the molecular recognition agents (e.g., α-hemolysin protein channel) are immobilized in this these corrals. This prevents the molecular recognition agents (e.g., protein channel) from diffusing away from the portion of the SLBM that is above a nanochannel opening/mouth.

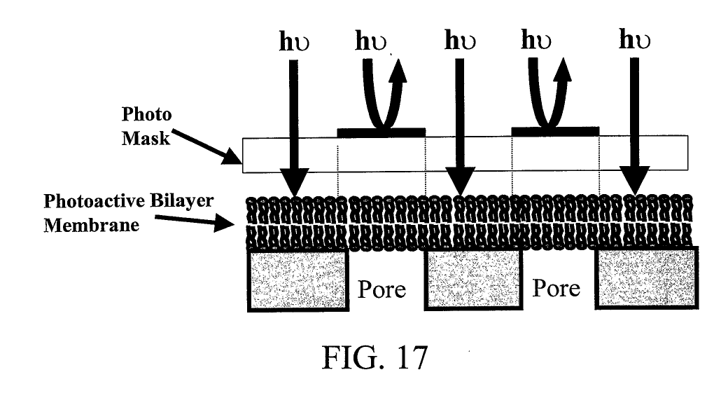

Protein corrals are prepared using a lipid labeled on its tail with a photoactive nitrobenzoxadiazole group. When an SLBM composed of this labeled lipid is photolyzed through a mask in the presence of IgG, the protein is covalently bound to the surface of the SLBM. Cooperative binding between adjacent IgG molecules causes them to cross-link to each other to form an immobile and impenetrable IgG deposit within the SLBM in the pattern determined by the photomask. The mask preferably consists of a transparent quartz plate with circular 200-nm Au pads on its surface. As shown in FIG. 17, these pads are in registry with the pores (dₜ also 200 nm) on the surface of the nanochannel support membrane. When the SLBM is photolyzed in the presence of IgG, cross-linked protein deposits will be formed around the pore mouths, leaving a corral of deposit-free pristine bilayer directly above each pore mouth.

Then, the desired molecular recognition agents (e.g., α-hemolysin protein channel) are each deposited into a corral above each nanochannel opening/mouth. In certain embodiments, the molecular recognition agents are deposited by simply exposing the SLBM to a solution of the molecular recognition agents. In a related embodiment, a nanoliter controllable micropipettor system is used to apply droplets of solutions containing the molecular recognition agents to the desired corral.

The second immobilization method entails lodging a portion of the molecular recognition agent, or nanocap (e.g., the stem of the α-hemolysin protein channel), into the opening/mouth of a conical nanochannel, as shown in FIG. 15A. The base of the cap will be covalently attached to the opening/mouth of the nanochannel to permanently lock the cap in place.

In certain embodiments, a very precise control over the tip diameter, dₜ, of the conical nanotube, is necessary. Taking αHL as the example molecular recognition agent/cap, if dₜ is too large (>10 nm), the molecular recognition agent/cap will pass through the nanochannel and not be lodged in the mouth. If dₜ is too small (<3 nm), the stem of the molecular recognition agent/cap will be too large to enter the mouth. A dₜ value of 5 nm would be ideal for this example (using αHL).

In one embodiment, gold (Au) is electrolessly plated along the nanochannel walls in track-etched membranes to obtain a gold-functionalized nanochannel within the support membrane (see, for example, Nishizawa, M. et al., Science, 268:700 (1995); Jirage, K. B. et al., Science, 278:655 (1997); Jirage, K. B. et al., Anal. Chem., 71:4913 (1999); and Lee, S. B. and Martin, C. R., Anal. Chem., 73:768 (2001)). By controlling the plating time, the inside diameter of the resulting nanotubes are very precisely controlled. Because of this ability to precisely control nanochannel diameter, support membranes containing conical Au nanochannels are used for the lodged-channel sensors (FIG. 15A).

Thiol-containing amino acid cysteine reacts with Au nanochannels to form a covalent S—Au bond, thus binding cysteine to the Au functionalized surface of the nanochannel (see, for example, Lee, S. B. and Martin, C. R., Anal. Chem., 73:768 (2001)). Accordingly, a simple route to covalently bind the αHL(+) in the opening/mouth of an Au nanochannel involves electrophoretically driving the stem-first into the mouth of a Au nanochannel. Because of the diameter of the opening of the nanochannel (e.g., dₜ=5 nm), the molecular recognition agent/cap cannot pass through the nanochannel. Where the bottom of molecular recognition agent/cap has surface cysteine residues, the residues would form Au—S bonds to the Au functionalized surface surrounding the opening/mouth of the nanochannel, thus locking the molecular recognition agent/cap in place (FIG. 15A). Furthermore, because the cysteines are at the bottom of the cap, they will be sterically prevented by the overlying cap from interacting with the Au surface until the stem is lodged into the mouth of the nanochannel.

Using the nanosensing systems based on the αHL channel, as described herein, divalent metal ions, including Co(II), Ni(II), Cu(II), Zn(II) can be detected and/or quantified. α-HL that have been genetically engineered to respond to divalent metal ions are used as the molecular recognition agent (see, for example, chemical and genetic engineering methods to prepare αHL mutants that respond to divalent metal ions (M²⁺) (Braha, O. et al., Chem. Biol., 4:497 (1997)), DNA (Howorka, S. et al., Proc. Natl. Acad. Sci. USA, 98:12996 (2001) and Howorka, S. et al., Nature Biotechnology, 19:636 (2001)), and the protein strepavidin (SA) (Movileanu, L. et al., Nature Biotechnology, 18:1091 (2000)). The magnitude and duration of the current block produced when each of these metal ions occupies the binding site in the engineered α-HL channel are measured.

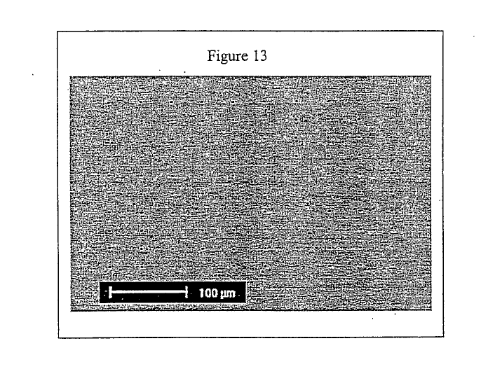

While the examples presented herein were for single-element (i.e., single channel) sensors, extending this concept to array-based sensors may also be possible in alternative embodiments of the present invention. FIG. 13 shows an array-based nanochannel membrane that is used in array-based nanochannel sensors, and such array-based nanochannel sensors are also a claim of this invention.

Alternatively, the present invention may utilize a plurality of channels, where each nanochannel is capable of detecting a different target analyte. As a result, the sensing device of the present invention may be used to detect a plurality of different analytes using a single sensing device.

All patents, patent applications, and publications referred to or cited herein are incorporated by reference in their entirety, including all figures and tables, to the extent that they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1-27. (canceled)

28. A sensing device for detecting the presence and/or concentration of a target analyte comprising:
a membrane having a single functionalized nanochannel, wherein the nanochannel comprises a passage;
a pH-response molecule; and
an assay means that produces a detection measurement response.

29. The device according to claim 28, wherein the nanochannel is functionalized with a lining material selected from the group consisting of: polytetrafluoroethylene (PTFE), polyethylene terephthalate, acrylonitrile-butadiene-styrene, acrylonitrile-methyl acetate copolymer, cellophane, ethyl cellulose, cellulose acetate, cellulose acetate butyrate, cellulose propionate, cellulose triacetate, polyethylene, polyethylene-vinyl acetate copolymers, ionomers, polyethylene-nylon copolymers, polypropylene, methyl pentene polymers, polyimide, polyvinyl fluoride, aromatic polysulfones, polypyrrole, polyaniline, polythiophene, TiS₂, TiO₂, silicon, germanium, graphite or various forms of graphitic carbon, gold, silver, copper, iron, steel, chromium, nickel, platinum, aluminum, copper, chromium oxide, zirconium, SiO₂, and Li⁺.

30. The device according to claim 29, wherein the nanochannel is functionalized with gold and the pH-response molecule is 2-mercaptopropionic acid.

31. The sensing device according to claim 28, wherein the nanochannel has a cross section in a shape selected from the group consisting of a: cone, hourglass, branched polygon, bicentric polygon, concave polygon, convex polygon, cyclic polygon, decagon, equiangular polygon, equilateral polygon, heptagon, hexagon, octagon, pentagon, decogram, octagram, hexagram, nonagram, pentagram, triangle, acute triangle, anticomplimentary triangle, equilateral triangle, isosceles triangle, obtuse triangle, right triangle, parallelogram, equilateral parallelogram, rectangle, rhomboid, Penrose tile, Penrose dart, Penrose kite, circle, Archimedes' circle, Bankoff circle, circumcircle, excircle, incircle, nine point circle, lune, semicircle, and ellipse.

32. The sensing device according to claim 28, wherein the nanochannel extends partially or entirely through the membrane.

33. The sensing device according to claim 28, wherein the detection measurement response is selected from the group consisting of: optical changes; electrical changes; changes in fluid flow; change in signal agent; and a combination thereof.

34. The sensing device according to claim 28, wherein the target analyte is selected from the group consisting of proteins, DNA, cells, spores, viruses, and a combination thereof.

35. The sensing device according to claim 28, wherein the assay means is selected from the group consisting of: fluorescence spectroscopy; UV-VIS absorption spectroscopy; Raman spectroscopy; Fourier transform infrared spectroscopy (FTIR); nuclear magnetic resonance (NMR); amperometry, cyclic voltammetry, potentiometry, and radiometric methods; and a combination thereof.

36. A sensing device for detecting the presence and/or concentration of a target analyte comprising:
a membrane having one nanochannel, wherein the nanochannel is functionalized and comprises: a passage, at least one opening, and at least one molecular recognition agent that has a high affinity for the target analyte; and
an assay means that produces a detection measurement response;
wherein when the target analyte binds to the at least one molecular recognition agent, a change in the detection measurement response indicates the presence and/or concentration of the target analyte.

37. The sensing device according to claim 36, wherein the molecular recognition agent is affixed near an opening of the nanochannel such that when the target analyte binds to the molecular recognition agent, the bound analyte blocks the opening and causes a change in the detection measurement response.

38. The sensing device according to claim 36, wherein the molecular recognition agent is affixed near an opening of the nanochannel, wherein the molecular recognition is bound to an entity that blocks the opening and passage of the nanochannel, such that when the target analyte preferentially binds to the molecular recognition agent, the entity disengages from the molecular recognition agent and opens the passage to cause a change in the detection measurement response.

39. The sensing device according to claim 36, wherein the molecular recognition agent is affixed in the passage of the nanochannel such that when the target analyte binds to the molecular recognition agent, the bound analyte blocks the passage and causes a change in the detection measurement response.

40. The sensing device according to claim 36, wherein the detection measurement response is selected from the group consisting of: optical changes; electrical changes; changes in fluid flow; change in signal agent; and a combination thereof.

41. The sensing device according to claim 36, wherein the nanochannel is functionalized with gold, the molecular recognition agent is biotin, and the target analyte is the protein Streptavidin.

42. The sensing device according to claim 36, wherein the nanochannel is functionalized with gold, the molecular recognition agent is a single-stranded DNA, and the target analyte is a complementary single-stranded DNA.

43. The sensing device according to claim 36, wherein the nanochannel is functionalized with gold, the molecular recognition agent is protein G, and the target analyte is immunoglobulin G.

44. The sensing device according to claim 36, wherein the nanochannel has a cross section in a shape selected from the group consisting of a: cone, hourglass, branched polygon, bicentric polygon, concave polygon, convex polygon, cyclic polygon, decagon, equiangular polygon, equilateral polygon, heptagon, hexagon, octagon, pentagon, decogram, octagram, hexagram, nonagram, pentagram, triangle, acute triangle, anticomplimentary triangle, equilateral triangle, isosceles triangle, obtuse triangle, right triangle, parallelogram, equilateral parallelogram, rectangle, rhomboid, Penrose tile, Penrose dart, Penrose kite, circle, Archimedes' circle, Bankoff circle, circumcircle, excircle, incircle, nine point circle, lune, semicircle, and ellipse.

45. The sensing device according to claim 36, wherein the target analyte is selected from the group consisting of proteins, DNA, cells, spores, viruses, and a combination thereof.

46. The sensing device according to claim 36, wherein the molecular recognition agent is a protein channel.

47. The sensing device according to claim 46, wherein the protein channel is αHL protein channel.

48. The sensing device according to claim 47, wherein the αHL protein channel is affixed over an opening of the nanochannel.

49. A sensing device for detecting the presence and/or concentration of a target analyte comprising:
a membrane having one nanochannel, wherein the nanochannel comprises a passage and at least one opening; and
an assay means that produces a detection measurement response;
wherein the nanochannel is functionalized, extends partially through the membrane, and comprises a signaling agent in the passage and a cap that blocks the at least one opening;
further wherein when the target analyte is present, the cap is released from the nanochannel and the signaling agent is released to cause a change in the detection measurement response, which indicates the presence and/or concentration of the target analyte.

50. The sensing device of claim 49, wherein at least one molecular recognition agent that preferentially binds to the target analyte is affixed to the cap, wherein the at least one molecular recognition agent is selected from an antibody, a protein, a single-stranded DNA molecule, a chelating agent, an aptamer, a biochemical ligand or receptor, or a combination thereof.

51. The sensing device according to claim 52, wherein the signaling agent is selected from the group consisting of: chromagens, chemiluminescers, radioactive labels, dyes that can be detected by optical absorbance, fluorophor molecules, quantum dots, redox-active molecules, and species that cause the pH of the solution to change raman-active substances.
